(19) United States
(12) Patent Application Publication
(10) Pub. No.: US 2011/0213137 A1
(43) Pub. Date: Sep. 1, 2011

(54) ISOTOPICALLY-LABELED GLYCANS

(75) Inventors: Carlos J. Bosques, Arlington, MA (US); Ian Christopher Parsons, Belmont, MA (US); Lakshmanan Thiruneelakantapillai, Boston, MA (US); Brian Edward Collins, Arlington, MA (US); Hetal Sarvaiya, Quincy, MA (US)

(73) Assignee: MOMENTA PHARMACEUTICALS, INC., CAMBRIDGE, MA (US)

(21) Appl. No.: 12/595,888

(22) PCT Filed: Apr. 15, 2008
(86) PCT No.: PCT/US08/60338

§ 371 Date: May 10, 2011

Related U.S. Application Data
(60) Provisional application No. 60/923,638, filed on Apr. 16, 2007.
(60) Provisional application No. 60/942,303, filed on Jun. 6, 2007.

(51) Int. Cl. C07B 59/00; G01N 30/72; C12Q 1/02

(52) U.S. Cl. 536/55.3; 436/94; 435/29

(57) ABSTRACT

The present disclosure provides methods of making isotopically-labeled glycans or a mixture of glycans (e.g., a glycan preparation) via reductive amination. Such methods are useful, for example, in the determination of the relative amounts of a glycan species present in two or more glycoprotein preparations using mass spectroscopy. In one aspect, the present disclosure provides a method of making an isotopically-labeled glycan by a glycan with an amine and an isotopically-labeled reducing agent to provide an isotopically-labeled aminated glycan. In certain embodiments, the amine is an isotopically-labeled amine.

### CROSS-REFERENCE TO RELATED APPLICATION

This application claims priority to U.S. provisional applications, Ser. No. 60/923,638, filed Apr. 16, 2007 and 60/942,303, filed Jun. 6, 2007, the entire contents of each of which are incorporated herein by reference.

### BACKGROUND

Alteration of glycosylation of a glycoprotein is known to affect the glycoprotein's biological activity and biophysical properties. For example, a glycoprotein's glycosylation pattern may affect its ability to fold correctly, its stability (e.g., resistance to proteolytic and/or other degradation), catalytic activity, immunogenicity, pharmacodynamic properties, pharmacokinetic properties, and/or its ability to properly interact with other molecules. Changes in the glycosylation pattern can also affect transport and/or targeting of the glycoprotein. Since glycosylation can vary in response to changes in manufacturing conditions, analysis of batch-to-batch homogeneity of a therapeutic glycoprotein preparation is important.

### SUMMARY

The present disclosure provides methods of isotopically-labeling a glycan or a mixture of glycans (e.g., a glycan preparation) via reductive amination. Such a method is useful, for example, in the determination of the relative amounts of a particular glycan present in two or more glycoprotein preparations using mass spectroscopy.

Thus, in one aspect, the present disclosure provides a method of making an isotopically-labeled aminated glycan comprising reacting a glycan with an amine and an isotopically-labeled reducing agent to provide an isotopically-labeled aminated glycan. The glycan which is reacted with the amine and the isotopically-labeled reducing agent necessarily comprises a reducing terminus (e.g., such as, for example, the —CHO group of a ring-opened glycan). In certain embodiments, the amine and the isotopically labeled reducing agent are reacted with the glycan simultaneously. In other embodiments, the amine and the isotopically-labeled reducing agent are reacted with the glycan sequentially. For example, in certain embodiments, the amine is reacted first to the glycan, and the isotopically-labeled reducing agent is reacted second. In certain embodiments, the isotopically-labeled reducing agent is reacted first to the glycan, and the amine is reacted second.

In one aspect, the isotopically-labeled reducing agent is deuterium (²H) labeled or tritium (³H) labeled. In certain embodiments, the isotopically-labeled reducing agent is deuterium (²H) labeled, and is selected from the group consisting of D₂-Pd/C, D₂-Raney Nickel; NaBD₄, pyridine-BD₃, α-picoline-BD₂; NaBD₃CN, NaBD(OCOCH₃)₃, and dialkylamino-BD₃. In certain embodiments, the dialkylamino-BD₃ is selected from dimethylamino-BD₃, diethylamino-BD₃ and dipropylamino-BD₃. In certain embodiments, the isotopically-labeled reducing agent is tritium (³H) labeled, and is selected from the group consisting of NaBT₄, pyridine-BT₃, α-picoline-BT₂; NaBT₃CN, NaBT(OCOCH₃)₃, and dialkylamino-BT₃. In certain embodiments, dialkylamino-BT₃ is selected from dimethylamino-BT₃, diethylamino-BT₃, dipropylamino-BT₃ and diisopropylamine-BD₃ and BT₃.

In certain embodiments, the isotopically-labeled product (e.g., the isotopically-labeled aminated glycan) comprises at least one ²H atom. In certain embodiments, the isotopically-labeled product comprises at least one ²H atom provided by transfer from the deuterium (²H) labeled reducing agent.

In certain embodiments, the isotopically-labeled product comprises at least one ³H atom. In certain embodiments, the isotopically-labeled product comprises at least one ³H atom provided by transfer from the tritium (³H) labeled reducing agent.

In certain embodiments, the amine is an isotopically-labeled amine. For example, in certain embodiments, the present disclosure provides a method of making an isotopically-labeled aminated glycan, comprising reacting a glycan with an isotopically-labeled amine and an isotopically labeled reducing agent to provide an isotopically-labeled aminated glycan.

In certain embodiments, the isotopically-labeled amine and the isotopically labeled reducing agent are reacted with the glycan simultaneously. In other embodiments, the isotopically-labeled amine and the isotopically-labeled reducing agent are reacted with the glycan sequentially. For example, in certain embodiments, the isotopically-labeled amine is reacted first to the glycan, and the isotopically-labeled reducing agent is reacted second (or afterwards). In certain embodiments, the isotopically-labeled reducing agent is reacted first to the glycan, and the isotopically-labeled amine is reacted second (or afterwards).

In certain embodiments, the isotopically-labeled amine comprises at least one substituent bearing an isotopic atom. Exemplary isotopic atoms include ²H, 3H, ¹³C, ¹⁸O, ¹⁵N, ¹⁸O, ³³S, ³⁴S, ³²P, ²⁹Si, ³⁰S, ¹¹B and ¹¹B. In certain embodiments, the nitrogen atom of the isotopically-labeled amine is an ¹⁵N atom.

According to the present method, the amine employed is either a primary amine, a secondary amine, or ammonia (NH₃). In one embodiment the amine employed is a primary amine or ammonia (NH₃).

In certain embodiments, the primary amine is substituted or unsubstituted, cyclic or acyclic, branched or unbranched alkyl amine; substituted or unsubstituted, cyclic or acyclic, branched or unbranched alkenyl amine; substituted or unsubstituted, cyclic or acyclic, branched or unbranched alkynyl amine; substituted or unsubstituted, cyclic or acyclic, branched or unbranched heteroalkyl amine; substituted or unsubstituted aryl amine; or substituted or unsubstituted heteroaryl amine.

In certain embodiments, the primary amine is a primary substituted or unsubstituted aryl amine or a primary substituted or unsubstituted heteroaryl amine. In certain embodiments, the primary substituted or unsubstituted aryl amine is selected from the group consisting of 2-aminobenzoic acid (2AA); 3-aminobenzoic acid (3AA); 4-aminobenzoic acid (4AA); 2-aminobenzamide (2AB); 3-aminobenzamide (3AB); 4-aminobenzamide (4AB); 2-aminobenzoic ethyl etser (2ABEE); 3-aminobenzoic ethyl etser (3ABEE); 4-aminobenzoic ethyl etser (4ABEE); 2-aminobenzonitrile (2ABN); 3-aminobenzonitrile (3ABN); 4-aminobenzonitrile (4ABN); methylanthranilate (MA); 8-aminonaphthalene-1,3,6-trisulfonic acid (ANTS); 2-aminonaphthalene-1,3,6-trisulfonate (ANT); 8-aminopyrene-1,3,6-trisulfonic acid (APTS); 9-fluorenylmethoxy-carbonyl-hydrazide (FMOC-hydrazide); 3,5-dimethylanthranilic acid; and 2-amino-4,5-dimethoxy-benzoic acid, and isotopically-labeled versions thereof For example, for any of the above listed amines, any one of the hydrogen atoms provided in the amine's chemical structure can be optionally isotopically labeled as ²H or ³H; any one of the carbon atoms provided in the amine's chemical structure can be optionally isotopically labeled as ¹³C; any one of the oxygen atoms provided in the amine's chemical structure can be optionally isotopically labeled as ¹⁸O; any one of the nitrogen atoms provided in the amine's chemical structure can be optionally isotopically labeled as ¹⁵N; and any one of the sulfur atoms provided in the amine's chemical structure can be optionally isotopically labeled as ³³S or ³⁴S.

In certain embodiments, the primary substituted or unsubstituted heteroaryl amine is selected from the group consisting of 2-aminopyridine (2AP); 2-amino(6-amido-biotinyl)pyridine (BAP); 6-aminoquinoline (6AQ); 3-(acetylamino)-6-aminoacridin (AA-AC); 2-aminoacridone (AMAC); and 7-aminomethyl-coumarin (AMC), and isotopically labeled versions thereof

In certain embodiments, the primary substituted or unsubstituted heteroaryl amine corresponds to the formula (I):

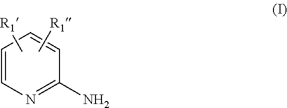

wherein

R₁′ and R₁″ are each independently —H, —NH₂, —NHR₂, —CONH₂, —COOH, —COR₃, —COOR₄, —SO₃, —SO—R₅ where n is 1 or 2, or a substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl, or substituted or unsubstituted heteroaryl, or when attached to adjacent carbon atoms R₁′ and R₁″ may be taken together with the atoms to which they are attached to form a 5- to 7-membered ring optionally containing a heteroatom selected from O, N or S;

R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl, or substituted or unsubstituted heteroaryl; and

wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸O; any one of the nitrogen atoms is optionally isotopically labeled as ¹⁵N; and any one of the sulfur atoms is optionally isotopically labeled as ³³S or ³⁴S.

In certain embodiments, the primary substituted or unsubstituted aryl amine corresponds to the formula (II):

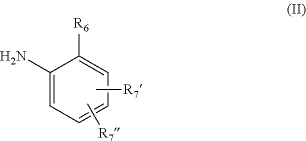

wherein:

R₆ is —H, —NH₂, —NHR₂, —CONH₂, —COOH, —COR₃, —COOR₄, —SO₃ or —SO—R₅ where n is 1 or 2;

R₇′ and R₇″ are each independently —H, —NH₂, —NHR₂, —CONH₂, —COOH, —COR₃, —COOR₄, —SO₃, —SO—R₅ where n is 1 or 2, or a substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl, or substituted or unsubstituted heteroaryl, or when attached to adjacent carbon atoms R₁ and R₁′ may be taken together with the atoms to which they are attached to form a 5- to 7-membered ring optionally containing a heteroatom selected from O, N or S;

R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group; and

wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸O; any one of the nitrogen atoms is optionally isotopically labeled as ¹⁵N; and any one of the sulfur atoms is optionally isotopically labeled as ³³S or ³⁴S.

The present disclosure also provides, in part, a method for determining the relative amount of a glycan species in at least two different glycan preparations, comprising steps of (i) reacting a first glycan preparation with a first amine and a first reducing agent under suitable reductive amination conditions to provide a first aminated glycan preparation; (ii) reacting a second glycan preparation with a second amine and a second reducing agent under suitable reductive amination conditions to provide a second aminated glycan preparation; (iii) combining an amount of the first aminated glycan preparation with an amount of the second aminated glycan preparation to provide a glycan mixture; (iv) analyzing the glycan mixture by mass spectrometry to provide a mass spectrum which includes at least one pair of peaks that corresponds to aminated versions of a glycan species from the first and second aminated glycan preparations; (v) determining the relative intensities of the peaks in the at least one pair of peaks; and (vi) determining, e.g., quantifying the relative amounts of the glycan species provided in the first and second glycan preparations in light of the relative intensities of the peaks.

### Definitions

Approximately, About: As used herein, the term “approximately” or “about,” as applied to one or more values of interest, refers to a value that is similar to a stated reference value. In certain embodiments, the terms “approximately” or “about” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the stated reference value.

Biological sample: The term “biological sample”, as used herein, refers to any solid or fluid sample obtained from, excreted by or secreted by any living cell or organism, including, but not limited to, tissue culture, bioreactors, human or animal tissue, plants, fruits, vegetables, single-celled microorganisms (such as bacteria and yeasts) and multicellular organisms. For example, a biological sample can be a biological fluid obtained from, e.g., blood, plasma, serum, urine, bile, seminal fluid, cerebrospinal fluid, aqueous or vitreous humor, or any bodily secretion, a transudate, an exudate (e.g., fluid obtained from an abscess or any other site of infection or inflammation), or fluid obtained from a joint (e.g., a normal joint or a joint affected by disease such as a rheumatoid arthritis, osteoarthritis, gout or septic arthritis). A biological sample can also be, e.g., a sample obtained from any organ or tissue (including a biopsy or autopsy specimen), can comprise cells (whether primary cells or cultured cells), medium conditioned by any cell, tissue or organ, tissue culture.

Cell-surface glycoprotein: As used herein, the term “cell-surface glycoprotein” refers to a glycoprotein, at least a portion of which is present on the exterior surface of a cell. In some embodiments, a cell-surface glycoprotein is a protein that is positioned on the cell surface such that at least one of the glycan structures is present on the exterior surface of the cell.

Cell-surface glycan: A “cell-surface glycan” is a glycan that is present on the exterior surface of a cell. In many embodiments of the disclosure, a cell-surface glycan is covalently linked to a polypeptide as part of a cell-surface glycoprotein. A cell-surface glycan can also be linked to a cell membrane lipid.

Glycan: As is known in the art and used herein “glycans” are sugars. Glycans can be monomers or polymers of sugar residues, but typically contain at least three sugars, and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′-sulfo N-acetylglucosamine, etc.). The term “glycan” includes homo and heteropolymers of sugar residues. The term “glycan” also encompasses a glycan component of a glycoconjugate (e.g., of a glycoprotein, glycolipid, proteoglycan, etc.). The term also encompasses free glycans, including glycans that have been cleaved or otherwise released from a glycoconjugate.

Glycan preparation: The term “glycan preparation” as used herein refers to a set of glycans obtained according to a particular production method. In some embodiments, glycan preparation refers to a set of glycans obtained from a glycoprotein preparation (see definition of glycoprotein preparation below).

Glycoconjugate: The term “glycoconjugate”, as used herein, encompasses all molecules in which at least one sugar moiety is covalently linked to at least one other moiety. The term specifically encompasses all biomolecules with covalently attached sugar moieties, including for example N-linked glycoproteins, O-linked glycoproteins, glycolipids, proteoglycans, etc.

Glycoform: The term “glycoform”, is used herein to refer to a particular form of a glycoconjugate. That is, when the same backbone moiety (e.g., polypeptide, lipid, etc) that is part of a glycoconjugate has the potential to be linked to different glycans or sets of glycans, then each different version of the glycoconjugate (i.e., where the backbone is linked to a particular set of glycans) is referred to as a “glycoform”.

Glycolipid: The term “glycolipid” as used herein refers to a lipid that contains one or more covalently linked sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may be comprised of one or more branched chains. In certain embodiments, sugar moieties may include sulfate and/or phosphate groups. In certain embodiments, glycoproteins contain O-linked sugar moieties; in certain embodiments, glycoproteins contain N-linked sugar moieties.

Glycoprotein: As used herein, the term “glycoprotein” refers to a protein that contains a peptide backbone covalently linked to one or more sugar moieties (i.e., glycans). As is understood by those skilled in the art, the peptide backbone typically comprises a linear chain of amino acid residues. In certain embodiments, the peptide backbone spans the cell membrane, such that it comprises a transmembrane portion and an extracellular portion. In certain embodiments, a peptide backbone of a glycoprotein that spans the cell membrane comprises an intracellular portion, a transmembrane portion, and an extracellular portion. In certain embodiments, methods comprise cleaving a cell surface glycoprotein with a protease to liberate the extracellular portion of the glycoprotein, or a portion thereof, wherein such exposure does not substantially rupture the cell membrane. The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. In certain embodiments, sugar moieties may include sulfate and/or phosphate groups. Alternatively or additionally, sugar moieties may include acetyl, glycolyl, propyl or other alkyl modifications. In certain embodiments, glycoproteins contain O-linked sugar moieties; in certain embodiments, glycoproteins contain N-linked sugar moieties. In certain embodiments, methods disclosed herein comprise a step of analyzing any or all of cell surface glycoproteins, liberated fragments (e.g., glycopeptides) of cell surface glycoproteins, cell surface glycans attached to cell surface glycoproteins, peptide backbones of cell surface glycoproteins, fragments of such glycoproteins, glycans and/or peptide backbones, and combinations thereof.

Glycosidase: The term “glycosidase” as used herein refers to an agent that cleaves a covalent bond between sequential sugars in a glycan or between the sugar and the backbone moiety (e.g. between sugar and peptide backbone of glycoprotein). In some embodiments, a glycosidase is an enzyme. In certain embodiments, a glycosidase is a protein (e.g., a protein enzyme) comprising one or more polypeptide chains. In certain embodiments, a glycosidase is a chemical cleavage agent.

Glycosylation pattern: As used herein, the term “glycosylation pattern” refers to the set of glycan structures present on a particular sample. For example, a particular glycoconjugate (e.g., glycoprotein) or set of glycoconjugates (e.g., set of glycoproteins) will have a glycosylation pattern. In some embodiments, reference is made to the glycosylation pattern of cell surface glycans. A glycosylation pattern can be characterized by, for example, the identities of glycans, amounts (absolute or relative) of individual glycans or glycans of particular types, degree of occupancy of glycosylation sites, etc., or combinations of such parameters.

Glycoprotein preparation: A “glycoprotein preparation”, as that term is used herein, refers to a set of individual glycoprotein molecules, each of which comprises a polypeptide having a particular amino acid sequence (which amino acid sequence includes at least one glycosylation site) and at least one glycan covalently attached to the at least one glycosylation site. Individual molecules of a particular glycoprotein within a glycoprotein preparation typically have identical amino acid sequences but may differ in the occupancy of the at least one glycosylation sites and/or in the identity of the glycans linked to the at least one glycosylation sites. That is, a glycoprotein preparation may contain only a single glycoform of a particular glycoprotein, but more typically contains a plurality of glycoforms. Different preparations of the same glycoprotein may differ in the identity of glycoforms present (e.g., a glycoform that is present in one preparation may be absent from another) and/or in the relative amounts of different glycoforms.

Isotopically labeled: As used herein, an “isotopically labeled” molecule bears an atom or set of atoms which have been isotopically-labeled by chemical or enzymatic methods. For example, an isotopically-labeled reducing agent or an isotopically-labeled amine may be isotopically-labeled by direct replacement of one or more atoms, either by chemical or enzymatic methods, with an isotopic label (e.g., a proton (¹H) with a deuterium (²H) atom; a proton (¹H) with a tritium (³H) atom; a ¹²C with a ¹³C atom; a ¹⁶O with a ¹⁸O atom; a ¹⁴N with a ¹⁵N atom; a ³¹P with a ³²P atom, and the like), or by chemical synthesis. Exemplary isotopic labels include, but are not limited to, ²H, ³H, ¹³C, ₁₈O, ¹⁵N, ¹⁸O, ³³S, ₃₄S, ³²P, ²⁹Si, ³⁰Si, ¹⁰B and ¹¹B. Furthermore, a glycan or glycan preparation may be “isotopically-labeled” (i.e., via conveyance/transfer of one or more isotopic atoms to the glycan moiety) by reaction with an isotopically-labeled reducing agent, or an isotopically-labeled reducing agent in conjunction with an isotopically-labeled amine. In the former case, the isotopic label is conveyed to the glycan via direct transfer from the reducing agent; in the latter case, the isotopic label is indirectly conveyed to the glycan by covalent attachment via the isotopically labeled amine.

Peak intensity: As used herein, the terms “peak intensity” refer to the magnitude of a particular peak within a mass spectrum. In one embodiment, the peak intensity is obtained by measuring the peak area. In one embodiment, the peak intensity is obtained by measuring the peak height.

Primary amine: A “primary amine” is an amine with one (non-hydrogen) substituent, and two hydrogen substituents. For example, a primary amine may be a substituted or unsubstituted, cyclic or acyclic, branched or unbranched alkyl amine; a substituted or unsubstituted, cyclic or acyclic, branched or unbranched alkenyl amine; a substituted or unsubstituted, cyclic or acyclic, branched or unbranched alkynyl amine; a substituted or unsubstituted, cyclic or acyclic, branched or unbranched heteroalkyl amine; a substituted or unsubstituted aryl amine; a substituted or unsubstituted heteroaryl amine, and the like, wherein the substituents alkyl, alkyenyl, alkynyl, heteroalkyl, aryl, and heteroaryl, are as defined herein.

Protease: The term “protease” as used herein refers to an agent that cleaves a peptide bond between sequential amino acids in a polypeptide chain. In some embodiments, a protease is an enzyme (i.e., a proteolytic enzyme). In certain embodiments, a protease is a protein (e.g., a protein enzyme) comprising one or more polypeptide chains. In certain embodiments, a protease is a chemical cleavage agent.

Protein: In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

Reductive amination: The terms “reductive amination” are art-understood terms which refer to the reaction between a primary amine with a carbonyl group (e.g., an aldehyde or a ketone) to form an imine, or the reaction between a secondary amine with a carbonyl group (e.g., an aldehyde or a ketone) to form an enamine, accompanied by the loss of one molecule of water and followed by reduction of the respective imine or enamine with a suitable reducing agent to provide an “aminated” product. Reductive amination may be indirect or direct. “Indirect reductive amination” is a two-step reaction in which an intermediate imine/enamine is reduced with a suitable reducing agent. For example, in the reductive amination of some aldehydes with primary amines where dialkylation is a problem, a stepwise procedure involving imine formation in MeOH followed by reduction with NaBH₄ is employed. “Direct reductive amination” is carried out in a “one pot” reaction with the imine/enamine formation and reduction occurring concurrently. Direct reductive amination is carried out with reducing agents that are more reactive toward imines/enamine than ketones, such as, for example, sodium cyanoborohydride (NaBH₃CN) or sodium triacetoxyborohydride (NaBH(OCOCH₃)₃). Exemplary reducing agents include, but are not limited to, NaBH₄, B₁₀H₁₄, pyridine-BH₃, α-picoline-BH₂/AcOH; H₂—Pd/C, H₂-Raney Nickel, NaBH₃CN, and NaBH(OCOCH₃)₃, an dialkylamine-BH₃ (e.g., dimethylamino-BH₃, diethylamino-BH₃ and dipropylamino-BH₃).

Secondary amine: A “secondary amine” is an amine with two (non-hydrogen) substituents., and one hydrogen substituent. For example, a secondary amine may be substituted with any combination of two alkyl, alkyenyl, alkynyl, heteroalkyl, aryl, and heteroaryl substituents, as defined herein. A secondary amine may be a bis-N-alkyl amine, bis-N-alkenyl amine, bis-N-alkynyl amine, bis-N-heteroalkyl amine, bis-N-aryl amine, N-alkyl-N-aryl-amine, N-alkyl-N-heteroalkylamine, N-alkyl-N-alkenylamine, N-alkyl-N-alkynylamine, N-alkenyl-N-arylamine, N-alkenyl-N-heteroarylamine, and the like.

### BRIEF DESCRIPTION OF THE DRAWING

FIG. 1 is a schematic depicting one embodiment of the comparative methods of the disclosure. As shown, two samples are taken from a single glycoprotein preparation. Glycans are then cleaved from the glycoproteins using two different methods (e.g., using different proteases and/or glycosidases) to produce two different glycan preparations. These are then labeled with labeling agents having different masses (e.g., d₀ vs. d₄ or d₅). Amounts of the two labeled glycan preparations are then mixed and analyzed by mass spectroscopy to produce a mass spectrum. Peaks corresponding to identical glycan species that originated from the two different glycan preparations are separated in the mass spectrum by the mass difference of the labeling agents (e.g., 4 or 5 Daltons). In another embodiment, two different glycoprotein preparations (e.g., from two different therapeutic protein products) are provided and glycans are cleaved from the glycoproteins using the same methods to produce two glycan preparations that are then labeled, mixed and analyzed by mass spectroscopy.

FIG. 2 is a schematic depicting another embodiment of the comparative methods of the disclosure. As shown, two samples are taken from different glycoprotein preparations. Glycans are then cleaved from the glycoproteins using two different methods (e.g., using different proteases and/or glycosidases) to produce two different glycan preparations. These are then labeled with labeling agents having different masses (e.g., d₀ vs. d₄ or d₅). Amounts of the two labeled glycan preparations are then mixed and analyzed by mass spectroscopy to produce a mass spectrum. Peaks corresponding to identical glycan species that originated from the two different glycan preparations are separated in the mass spectrum by the mass difference of the labeling agents (e.g., 4 or 5 Daltons). In another embodiment, two different glycoprotein preparations (e.g., from two different therapeutic protein products) are provided and glycans are cleaved from the glycoproteins using the same methods to produce two glycan preparations that are then labeled, mixed and analyzed by mass spectroscopy.

FIG. 3 shows exemplary mass spectra generated in the quantification of isotopically labeled glycans present in a glycan preparation using methods described herein. When the different isotopically labeled glycan preparations are combined (bottom figure), their relative quantities can be directly compared by calculating the ratios between the d₀ and d₄ labeled species.

FIG. 4 shows exemplary mass spectra demonstrating the relative quantitation of glycan species released from different glycoprotein samples using the method described in FIG. 2. The spectra show the relative amounts obtained for a specific sialylated N-linked glycan derived from two different protein samples when these are combined in approximately 1:1 ratio (top spectrum), 1:5 ratio (middle spectrum) or 1:10 ratio (bottom spectrum).

FIG. 5 provides chromatographic and mass spectrometry data generated from glycan preparations according to the Scheme in FIG. 1. The chromatogram shown in A represents the separation of glycans species of two combined samples containing d₀ and d₄-labeled glycans. The mass spectra shown in B illustrates the relative quantitation of one particular glycan species present in the two samples based on the d₀/d₄ ratio.

### DETAILED DESCRIPTION OF CERTAIN EMBODIMENTS

The present disclosure provides methods of isotopically-labeling a glycan or a mixture of glycans via reductive amination using an isotopically labeled reducing agent. Such methods are useful, for example, in the determination of relative amounts of a particular glycan present in a glycan or glycoprotein preparation using mass spectroscopy.

### Isotopically-Labeling Glycans

In one aspect, the present disclosure provides a method of making an isotopically-labeled glycan by reacting a glycan with an amine and an isotopically-labeled reducing agent to provide an isotopically labeled aminated glycan. The glycan which is reacted with the amine and the isotopically-labeled reducing agent necessarily comprises a reducing terminus (e.g., for example, a —CHO group of a ring-opened glycan).

In certain embodiments, the amine is an isotopically labeled amine. For example, in certain embodiments, the present disclosure provides a method of making an isotopically-labeled glycan by reacting a glycan with an isotopically labeled amine and an isotopically-labeled reducing agent to provide an isotopically labeled aminated glycan.

In certain embodiments, the amine and the isotopically labeled reducing agent are reacted with the glycan simultaneously. In other embodiments, the amine and the isotopically-labeled reducing agent are reacted with the glycan sequentially. For example, in certain embodiments, the amine is reacted first to the glycan, and the isotopically-labeled reducing agent is reacted second. In certain embodiments, the isotopically-labeled reducing agent is reacted first to the glycan, and the amine is reacted second.

Reductive amination may also be performed with nitro-groups, nitroso-groups, and azo-groups as in situ generated amines. In this particular case, the group in question is reduced under the reductive reaction conditions employed to provide a primary amine, which, in turn, reacts with a glycan or glycan preparation via reductive amination to provide an isotopically-labeled product.

For example, the present disclosure provides a method of making an isotopically-labeled glycan by reacting an amine, generated in situ, with a glycan using an isotopically-labeled reducing agent to provide an isotopically-labeled aminated glycan.

In certain embodiments, the isotopically-labeled reducing agent is deuterium (²H) labeled. In certain embodiments, the deuterium (²H) labeled reducing agent is selected from the group consisting of D₂-Pd/C, D₂-Raney Nickel, NaBD₄, B₁₀D₁₄, pyridine-BD₃, α-picoline-BD₂; NaBD₃CN, NaBD(OCOCH₃)₃, and dialkylamino-BD₃. In certain embodiments, dialkylamino-BD₃ is selected from dimethylamino-BD₃, diethylamino-BD₃ and dipropylamino-BD₃.

In certain embodiments, the isotopically-labeled reducing agent is tritium (³H) labeled. In certain embodiments, the tritium (³H) labeled reducing agent is selected from the group consisting of NaBT₄, B₁₀T₁₄, pyridine-BT₃, α-picoline-BT₂; NaBT₃CN, NaBT(OCOCH₃)₃, and dialkylamino-BT₃. In certain embodiments, dialkylamino-BT₃ is selected from dimethylamino-BT₃, diethylamino-BT₃ and dipropylamino-BT₃.

In certain embodiments, the amine employed in the reductive amination reaction is also isotopically-labeled. In certain embodiments, the isotopically-labeled amine comprises at least one substituent bearing an isotopic atom. Exemplary isotopic atoms include ²H, ³H, ¹³C, ¹⁸O, ¹⁵N, ¹⁸O, ³³S, ³⁴S, ³²P, ²⁹Si, ³⁰Si, ¹⁰B and ¹¹B. According to such embodiments, the use of an isotopically-labeled reducing agent provides a mechanism for further increasing the mass of the isotopically-labeled aminated glycan (and thus creating a greater mass difference with a non-isotopically labeled counterpart). In certain embodiments, the nitrogen atom of the isotopically-labeled amine is an ¹⁵N atom. In certain embodiments, the isotopically-labeled amine comprises at least one substituent bearing an ²H atom. In certain embodiments, the isotopically-labeled amine comprises at least one substituent bearing an ³H atom. In certain embodiments, the isotopically-labeled amine comprises at least one substituent bearing an ¹³C atom. In certain embodiments, the isotopically-labeled amine comprises at least one substituent bearing an ¹⁵N atom. In certain embodiments, the isotopically-labeled amine comprises at least one substituent bearing an ¹⁸O atom. In certain embodiments, the isotopically-labeled amine comprises at least one substituent bearing an ³³S atom. In certain embodiments, the isotopically-labeled amine comprises at least one substituent bearing an ³⁴S atom. In certain embodiments, the isotopically-labeled amine comprises at least one substituent bearing a ³²P atom. In certain embodiments, the isotopically-labeled amine comprises at least one substituent bearing an ²⁹Si atom. In certain embodiments, the isotopically-labeled amine comprises at least one substituent bearing an ³⁰Si atom.

In one set of embodiments, the isotopically-labeled amine includes one isotopic label. In other embodiments, the isotopically-labeled amine may include more than one isotopic label. For example, the isotopically-labeled amine may include two different isotopic labels (e.g., ²H and ¹³C). Alternatively, the isotopically-labeled amine may include two or more copies of the same isotopic label (e.g., ²H).

In certain embodiments, the isotopically-labeled product (e.g., an isotopically-labeled aminated glycan) comprises at least one ²H atom. In certain embodiments, the isotopically-labeled product comprises at least one ²H atom provided from a deuterium (²H) labeled reducing agent. In certain embodiments, the isotopically-labeled product comprises at least one ³H atom. In certain embodiments, the isotopically-labeled product comprises at least one ³H atom provided from a tritium (³H) labeled reducing agent.

In certain embodiments, the isotopically-labeled product comprises at least two ²H atoms. In certain embodiments, the isotopically-labeled product comprises at least one ²H atom provided from a deuterium (²H) labeled reducing agent, and at least one ²H atom provided from a deuterium (²H) labeled amine.

In certain embodiments, the isotopically-labeled product comprises at least three ²H atoms. In certain embodiments, the isotopically-labeled product comprises at least one ²H atom provided from a deuterium (²H) labeled reducing agent, and at least two ²H atoms provided from a deuterium (²H) labeled amine.

In certain embodiments, the isotopically-labeled product comprises at least four ²H atoms. For example, in certain embodiments, the isotopically-labeled product comprises at least one ²H atom provided from a deuterium (²H) labeled reducing agent, and at least three ²H atoms provided from a deuterium (²H) labeled amine.

In certain embodiments, the isotopically-labeled product comprises at least five ²H atoms. For example, in certain embodiments, the isotopically-labeled product comprises at least one ²H atom provided from a deuterium (²H) labeled reducing agent, and at least four ²H atoms provided from a deuterium (²H) labeled amine.

In certain embodiments, the isotopically-labeled product comprises at least six ²H atoms. In certain embodiments, the isotopically-labeled product comprises at least seven ²H atoms.

According to the present method, the amine is a primary amine, a secondary amine, or ammonia (NH₃). In one embodiment the amine employed is a primary amine or ammonia (NH₃).

In certain embodiments, the primary amine is substituted or unsubstituted, cyclic or acyclic, branched or unbranched alkyl amine; substituted or unsubstituted, cyclic or acyclic, branched or unbranched alkenyl amine; substituted or unsubstituted, cyclic or acyclic, branched or unbranched alkynyl amine; substituted or unsubstituted, cyclic or acyclic, branched or unbranched heteroalkyl amine; substituted or unsubstituted aryl amine; or substituted or unsubstituted heteroaryl amine.

In certain embodiments, the primary amine is a primary substituted or unsubstituted aryl amine or a primary substituted or unsubstituted heteroaryl amine. In certain embodiments, the primary substituted or unsubstituted aryl amine is selected from the group consisting of 2-aminobenzoic acid (2AA); 3-aminobenzoic acid (3AA); 4-aminobenzoic acid (4AA); 2-aminobenzamide (2AB); 3-aminobenzamide (3AB); 4-aminobenzamide (4AB); 2-aminobenzoic ethyl etser (2ABEE); 3-aminobenzoic ethyl etser (3ABEE); 4-aminobenzoic ethyl etser (4ABEE); 2-aminobenzonitrile (2ABN); 3-aminobenzonitrile (3ABN); 4-aminobenzonitrile (4ABN); methylanthranilate (MA); 8-aminonaphthalene-1,3,6-trisulfonic acid (ANTS); 2-aminonaphthalene-1,3,6-trisulfonate (ANT); 8-aminopyrene-1,3,6-trisulfonic acid (APTS); 9-fluorenylmethoxy-carbonyl-hydrazide (FMOC-hydrazide); 3,5-dimethylanthranilic acid; and 2-amino-4,5-dimethoxy-benzoic acid, or an isotopically-labeled version thereof. For example, for any of the aforementioned amines, any one of the hydrogen atoms provided in the amine's chemical structure can be optionally isotopically labeled as ²H or ³H; any one of the carbon atoms provided in the amine's chemical structure can be optionally isotopically labeled as ¹³C; any one of the oxygen atoms provided in the amine's chemical structure can be optionally isotopically labeled as ¹⁸O; any one of the nitrogen atoms provided in the amine's chemical structure can be optionally isotopically labeled as ¹⁵N; and any one of the sulfur atoms provided in the amine's chemical structure can be optionally isotopically labeled as ³³S or ³⁴S.

In certain embodiments, the primary substituted or unsubstituted heteroaryl amine is selected from the group consisting of 2-aminopyridine (2AP); 2-amino(6-amido-biotinyl)pyridine (BAP); 6-aminoquinoline (6AQ); 3-(acetylamino)-6-aminoacridin (AA-AC); 2-aminoacridone (AMAC); and 7-aminomethyl-coumarin (AMC), or an isotopically labeled version thereof

In certain embodiments, the primary substituted or unsubstituted heteroaryl amine corresponds to the formula (I):

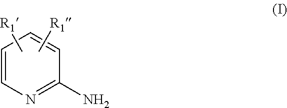

wherein

R₁′ and R₁″ are each independently —H, —NH₂, —NHR₂, —CONH₂, —COOH, —COR₃, —COOR₄, —SO₃, —SOₙR₅ where n is 1 or 2, or a substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group, or when attached to adjacent carbon atoms R₁′ and R₁″ may be taken together with the atoms to which they are attached to form a 5- to 7-membered ring optionally containing a heteroatom selected from O, N or S;

R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group; and

wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸O; any one of the nitrogen atoms is optionally isotopically labeled as ¹⁵N; and any one of the sulfur atoms is optionally isotopically labeled as ³³S or ³⁴S.

In certain embodiments, the primary substituted or unsubstituted aryl amine corresponds to the formula (II):

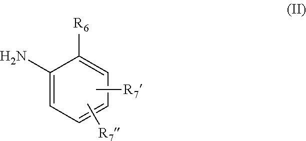

wherein:

R₆ is —H, —NH₂, —NHR₂, —CONH₂, —COOH, —COR₃, —COOR₄, —SO₃ or —SO—R₅ where n is 1 or 2;

R₂, R₃, R₄ and R₅ are each independently —H or substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group;

R₇′ and R₇″ are each independently —H, —NH₂, —NHR₂, —CONH₂, —COOH, —COR₃, —COOR₄, —SO₃, —SO—R₅ where n is 1 or 2, or an substituted or unsubstituted, cyclic or acyclic, branched or unbranched alkyl, substituted or unsubstituted, cyclic or acyclic, branched or unbranched alkenyl, substituted or unsubstituted, cyclic or acyclic, branched or unbranched alkynyl, substituted or unsubstituted, cyclic or acyclic, branched or unbranched heteroalkyl, substituted or unsubstituted aryl or substituted or unsubstituted heteroaryl group, or when attached to adjacent carbon atoms R₁ and R₁′ may be taken together with the atoms to which they are attached to form a 5- to 7-membered ring optionally containing a heteroatom selected from O, N or S; and

wherein any one of the hydrogen atoms is optionally isotopically labeled as ²H or ³H; any one of the carbon atoms is optionally isotopically labeled as ¹³C; any one of the oxygen atoms is optionally isotopically labeled as ¹⁸O; any one of the nitrogen atoms is optionally isotopically labeled as ¹⁵N; and any one of the sulfur atoms is optionally isotopically labeled as ³³S or ³⁴S.

In certain embodiments, R₁′ and R₁″ are each independently —H, —NH₂, —NHR₂, —CONH₂, —COOH, —COR₃, —COOR₄, —SO₃, —SO—R₅ where n is 1 or 2, or unsubstituted, cyclic or acyclic alkyl; unsubstituted, cyclic or acyclic alkenyl; unsubstituted, cyclic or acyclic alkynyl; unsubstituted, cyclic or acyclic heteroalkyl; unsubstituted aryl, or unsubstituted heteroaryl group, or when attached to adjacent carbon atoms R₁′ and R₁″ may be taken together with the atoms to which they are attached to form a 5- to 7-membered ring optionally containing a heteroatom selected from O, N or S.

In certain embodiments, R₂, R₃, R₄ and R₅ are each independently —H or unsubstituted, cyclic or acyclic alkyl; unsubstituted, cyclic or acyclic alkenyl; unsubstituted, cyclic or acyclic alkynyl; unsubstituted, cyclic or acyclic heteroalkyl, unsubstituted aryl or unsubstituted heteroaryl group.

In certain embodiments, R₆ is —H.

In certain embodiments, R₇′ and R₇″ are each, independently, —H, —NH₂, —NHR₂, —CONH₂, —COOH, —COR₃, —COOR₄, —SO₃, —SO—R₅ where n is 1 or 2, or unsubstituted, cyclic or acyclic alkyl, unsubstituted, cyclic or acyclic alkenyl, unsubstituted, cyclic or acyclic alkynyl, unsubstituted, cyclic or acyclic heteroalkyl, unsubstituted aryl or unsubstituted heteroaryl group, or when attached to adjacent carbon atoms R₁ and R₁′ may be taken together with the atoms to which they are attached to form a 5- to 7-membered ring optionally containing a heteroatom selected from O, N or S.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

In general, the term “substituted” refers to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein and any combination thereof that results in the formation of a stable moiety. The present disclosure contemplates any and all such combinations in order to arrive at a stable substituent/moiety. For purposes of this disclosure, heteroatoms such as nitrogen may have hydrogen substituents and/or any suitable substituent as described herein which satisfy the valencies of the heteroatoms and results in the formation of a stable moiety. The term “stable moiety,” as used herein, preferably refers to a moiety which possess stability sufficient to allow manufacture, and which maintains its integrity for a sufficient period of time to be useful for the purposes detailed herein.

The term “alkyl,” as used herein, refers to saturated, cyclic or acyclic, branched or unbranched, substituted or unsubstituted hydrocarbon radicals derived from a hydrocarbon moiety containing between one and twenty carbon atoms by removal of a single hydrogen atom. In some embodiments, the alkyl group contains 1-20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more sustitutents. Alkyl group substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, alkyloxy, aryloxy, alkyloxyalkyl, azido, oxo, cyano, halo, isocyano, nitro, nitroso, azo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; haloalkyl, alkoxyalkyl, substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “cycloalkyl” refers to a cyclic alkyl group, as defined herein. Cycloalkyl groups include cyclopropyl, cyclobutyl, cyclopentyl, cyclhexyl, cycloheptyl, cyclooctyl, cyclononyl, cyclodecyl, cycloundecyl, cyclododecyl, and the like, which may bear one or more sustitutents. Cycloalkyl group substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “alkenyl,” as used herein, denotes a monovalent group derived from a cyclic or acyclic, branched or unbranched, substituted or unsubstituted hydrocarbon moiety having at least one carbon-carbon double bond by the removal of a single hydrogen atom. In certain embodiments, the alkenyl group contains 2-20 carbon atoms. In some embodiments, the alkenyl group contains 2-15 carbon atoms. In another embodiment, the alkenyl group employed contains 2-10 carbon atoms. In still other embodiments, the alkenyl group contains 2-8 carbon atoms. In yet another embodiments, the alkenyl group contains 2-5 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “alkynyl,” as used herein, refers to a monovalent group derived from a cyclic or acyclic, branched or unbranched, substituted or unsubstituted hydrocarbon having at least one carbon-carbon triple bond by the removal of a single hydrogen atom. In certain embodiments, the alkynyl group contains 2-20 carbon atoms. In some embodiments, the alkynyl group contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 2-propynyl(propargyl), 1-propynyl, and the like, which may bear one or more substituents. Alkynyl group substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “heteroalkyl,” as used herein, refers to an alkyl moiety, as defined herein, which includes saturated, cyclic or acyclic, branched or unbranched, substituted or unsubstituted hydrocarbon radicals, which contain one or more oxygen, sulfur, nitrogen, phosphorus, or silicon atoms, e.g., in place of carbon atoms. In certain embodiments, hetereoalkyl moieties are substituted by independent replacement of one or more of the hydrogen atoms thereon with one or more substituents. Heteroalkyl substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

As used herein the term “haloalkyl” designates a CₙH₂ₙ₊₁ group having from one to 2n+1 halogen atoms which may be the same or different. Examples of haloalkyl groups include CF₃, CH₂Cl, C₂H₃BrCl, C₃H₅F₂, or the like. Similarly, the term haloalkoxy designates an OCₙH₂ₙ₊₁ group having from one to 2n+1 halogen atoms which may be the same or different.

The term “alkoxyalkyl”, as used herein, refers to an alkyl group as hereinbefore defined substituted with at least one alkyloxy group.

The term “cycloheteroalkyl,” as used herein, refers to a cyclic heteroalkyl group as defined herein. A cycloheteroalkyl group refers to a fully saturated 3- to 10-membered ring system, which includes single rings of 3 to 8 atoms in size. These cycloheteroalkyl rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term cycloheteroalkyl refers to a 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Examples of cycloheteroalkyl ring systems included in the term as designated herein are the following rings wherein X₁ is NR′, O or S, and R′ is H or an optional substituent as defined herein:

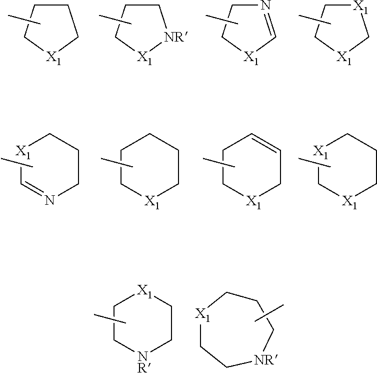

Exemplary cycloheteroalkyls include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group)

The term “aryl,” as used herein, refer to stable aromatic mono- or polycyclic ring system having 3-20 ring atoms, of which all the ring atoms are carbon, and which may be substituted or unsubstituted. In certain embodiments, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, substituted or unsubstituted thio, haloalkyl, alkyloxy, aryloxy, alkyloxyalkyl, azido, cyano, halo, isocyano, nitro, nitroso, azo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group)

The term “amino,” as used herein, refers to a group of the formula (—NH₂). A “substituted amino” refers either to a mono-substituted amino (—NHR^{h}) or a di-substituted amino (—NR^{h}₂), wherein the R^{h} substituent is any substitutent as described herein that results in the formation of a stable moiety (e.g., a cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, substituted or unsubstituted amino, substituted or unsubstituted hydroxy, haloalkyl, alkyloxy, aryloxy, alkyloxyalkyl, azido, cyano, halo, oxo, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group). In certain embodiments, the R^{h} substituents of the di-substituted amino group (—NR^{h}₂) form an optionally substituted 5- to 6-membered cycloheteroalkyl ring. A dialkylamino group is a di-substituted amino group, as defined herein, wherein each R^{h} is, independently, an alkyl group, or two R^{h} alkyl groups are joined together to form a 5- to 6-membered ring. Exemplary dialkylamino groups include dimethylamino, di-ethylamino, di-propylamino, di-isopropylamino, ethylisopropylamino, pyrrolidinyl, piperidinyl, and the like.

The term “hydroxy,” or “hydroxyl,” as used herein, refers to a group of the formula (—OH). A “substituted hydroxyl” refers to a group of the formula (—ORⁱ) wherein Rⁱ can be any substitutent which results in a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group)

The term “thio” or “thiol” as used herein, refers to a group of the formula (—SH). A “substituted thiol” refers to a group of the formula (—SR^{r}), wherein R^{r} can be any substitutent which results in a stable moiety (e.g., cyclic or acyclic, branched or unbranched, substituted or unsubstituted alkyl, cyclic or acyclic, branched or unbranched, substituted or unsubstituted alkenyl, cyclic or acyclic, branched or unbranched, substituted or unsubstituted alkynyl, substituted or unsubstituted aryl, substituted or unsubstituted heteroaryl, —CONH₂, —COOH, —COR₃, —COOR₄, —SO₃, —SOₙR₅, wherein n is 1 or 2, and R₂, R₃, R₄ and R₅ are each independently —H or substituted or unsubstituted, cyclic or acyclic, branched or unbranched alkyl; substituted or unsubstituted, cyclic or acyclic, branched or unbranched alkoxyalkyl; substituted or unsubstituted, cyclic or acyclic, branched or unbranched alkenyl; substituted or unsubstituted, cyclic or acyclic, branched or unbranched alkynyl; substituted or unsubstituted, cyclic or acyclic, branched or unbranched cycloalkyl, substituted or unsubstituted, cyclic or acyclic, branched or unbranched cycloheteroalkyl, substituted or unsubstituted aryl or substituted or unsubstituted heteroaryl group).

The term “alkyloxy” refers to a “substituted hydroxyl” of the formula (—ORⁱ), wherein Rⁱ is an optionally substituted alkyl group, as defined herein, and the oxygen moiety is directly attached to the parent molecule. The term “alkylthioxy” refers to a “substituted thiol” of the formula (—SR^{r}), wherein R^{r} is an optionally substituted alkyl group, as defined herein, and the sulfur moiety is directly attached to the parent molecule. The term “alkylamino” refers to a “substituted amino” of the formula (—NR^{h}₂), wherein R^{h} is, independently, a hydrogen or an optionally substituted alkyl group, as defined herein, and the nitrogen moiety is directly attached to the parent molecule.

The term “aryloxy” refers to a “substituted hydroxyl” of the formula (—ORⁱ), wherein Rⁱ is an optionally substituted aryl group, as defined herein, and the oxygen moiety is directly attached to the parent molecule. The term “arylamino,” refers to a “substituted amino” of the formula (—NR^{h}₂), wherein R^{h} is, independently, a hydrogen or an optionally substituted aryl group, as defined herein, and the nitrogen moiety is directly attached to the parent molecule. The term “arylthioxy” refers to a “substituted thiol” of the formula (—SR^{r}), wherein R^{r} is an optionally substituted aryl group, as defined herein, and the sulfur moiety is directly attached to the parent molecule.

The term “alkyloxyalkyl” or “alkoxyalkyl” as used herein refers to an alkyloxy group, as defined herein, attached to an alkyl group attached to the parent molecule.

The term “azido,” as used herein, refers to a group of the formula (—N₃).

The term “cyano,” as used herein, refers to a group of the formula (—CN).

The terms “halo” and “halogen” as used herein refer to an atom selected from fluorine (fluoro, —F), chlorine (chloro, —Cl), bromine (bromo, —Br), and iodine (iodo, —I).

The term “isocyano,” as used herein, refers to a group of the formula (—NC).

The term “nitro,” as used herein, refers to a group of the formula (—NO₂).

The term “nitroso,” as used herein, refers to a group of the formula (—N═O).

The term “azo,” as used herein, refers to a group of the formula (—N₂).

The term “oxo,” as used herein, refers to a group of the formula (═O).

Reductive amination of a glycan proceeds via reaction of an amine with the glycan's reducing (—CHO) end in the presence of a suitable reducing agent (see exemplary reaction, Scheme 1, with suitable reducing agent NaCNBD₃). One of ordinary skill in the art will appreciate that a wide variety of reaction conditions may be employed to promote a reductive amination reaction, therefore, a wide variety of reaction conditions are envisioned; see generally, March's Advanced Organic Chemistry: Reactions, Mechanisms, and Structure, M. B. Smith and J. March, 5^{th} Edition, John Wiley & Sons, 2001, and Comprehensive Organic Transformations, R. C. Larock, 2^{nd} Edition, John Wiley & Sons, 1999; and see specifically, Abdel-Magid et al., J. Org. Chem., (1996) 61:3849-3862 (e.g., use of sodium triacetoxyborohydride; NaBH₄/MeOH); McLaughlin et al., Org. Lett., (2006) 8:3307-3310 (e.g., an effective reductive alkylation of electron-deficient o-chloroarylamines); Mizuta et al., J. Org. Chem. (2005) 70:2195-2199 (e.g., methodology for the reductive alkylation of secondary amines with aldehydes and Et₃SiH using an iridium catalyst; use of an environmentally friendly reducing reagent polymethylhydrosiloxane (PMHS)); Menche et al., Org. Lett. (2006) 8:741-744 (e.g., direct reductive amination using selective imine activation with thiourea followed by transfer hydrogenation); Kumpaty et al, Synthesis (2003) 2206-2210 (e.g., a reductive mono-N-alkylation of primary amines with carbonyl compounds in the presence of Ti(i-PrO)₄ and NaBH₄); Miriyala et al., Tetrahedron (2004) 60:1463-1471 (e.g., reductive alkylation of ammonia with aldehydes); Sato et al., Tetrahedron (2004) 60:7899-7906 (e.g., one-pot reductive amination of aldehydes and ketones with amines using α-picoline-borane as a reducing agent in the presence of small amounts of AcOH, MeOH, and/or H₂O, and in neat conditions); and Bae et al., Chem. Commun. (2000) 1857-1858 (e.g., reduction of nitrobenzenes followed by reductive amination with decaborane (B₁₀H₁₄) in the presence of 10% Pd/C); the entirety of each of which is hereby incorporated herein by reference.

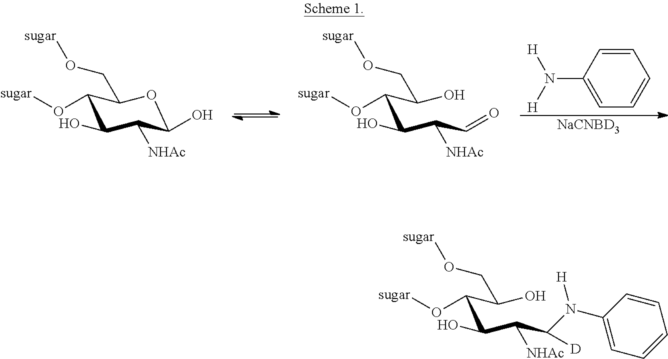

In certain embodiments, the reaction is carried out under one or more suitable reductive amination conditions. Suitable reductive amination conditions include, but are not limited to, the type of medium, the pH of the medium, the reducing agent (as detailed above), and/or use of a catalyst.

In certain embodiments, the reductive amination reaction is carried out in a suitable medium. A suitable medium is a solvent or a solvent mixture that, in combination with the combined reacting partners and reagents, facilitates the progress of the reaction therebetween. A suitable medium may solubilize one or more of the reaction components, or, alternatively, the suitable medium may facilitate the suspension of one or more of the reaction components; see, generally, Smith and March, March's Advanced Organic Chemistry, 5^{th} Edition, John Wiley & Sons, Inc., New York, 2001. Solvents which may be used to facilitate the reductive amination reaction include, but are not limited to, ethers, halogenated hydrocarbons, aromatic solvents, esters, or mixtures thereof. In certain embodiments, the solvent is diethyl ether, dioxane, tetrahydrofuran (THF), dichloromethane (DCM), dichloroethane (DCE), dimethylsulfoxide (DMSO), dimethyformamide (DMF), chloroform, toluene, benzene, ethyl acetate, isopropyl acetate, or a mixture thereof.

In certain embodiments, the solvent medium further comprises one or more hydrogen-donating additives, such as an organic alcohol and/or an acid. Exemplary organic alcohols include, but are not limited to, methanol, ethanol, isopropanol, or t-butanol. Exemplary hydrogen donating acids include, but are not limited to, hydrogen chloride, hydrogen bromide, hydrogen iodide, hydrogen fluoride, formic acid, toluenesulfonic acid (TsOH), trifluoroacetic acid (TFA), and acetic acid (AcOH). In certain embodiments, the pH of the reaction solution is acidic. In certain embodiments, the pH of the reaction solution is between about 4 to about 6.

In certain embodiments, the solvent medium is buffered (e.g., contains a buffer such as NaOAc—AcOH). In certain embodiments, the pH of the buffered reaction solution is acidic. In certain embodiments, the pH of the buffered reaction solution is between about 4 to about 6.

In certain embodiments, the reductive amination reaction also comprises a catalyst, such as a Lewis acid catalyst or a transition metal catalyst. Exemplary Lewis acid catalysts include, but are not limited to, Ti(i-PrO)₄, AlCl₃, BCl₃, BBr₃, and FeCl₃. Exemplary transition metal catalysts include, but are not limited to, nickel, zinc, ruthenium, rhodium, palladium, platinum, gold, and mercury. In certain embodiments, the transition metal catalyst is a palladium catalyst. In certain embodiments, the palladium catalyst is palladium-on-carbon. In certain embodiments, the reductive amination reaction is conducted under hydrogen gas or deuterium gas pressure (e.g., between about 5 to about 80 psi).

### Mass Spectroscopic Analysis and Quantification

The present disclosure also provides a quantification method for determining the relative amount of a glycan provided in different glycan preparations using mass spectroscopy. For example, the glycan preparations may be obtained from a glycoprotein preparation, which in turn, is obtained according to a particular production method (e.g., such as a production method used to cleave one or more glycans from a cell surface glycoprotein or a glycoprotein secreted from a cell). Different glycoprotein preparations may differ in the identity and/or in the relative amounts of glycans present (e.g., a glycan present in one preparation may be absent from another) based on the differences between production methods. In many embodiments of the disclosure, strategies are employed to compare different preparations of the same protein (i.e., the same amino acid sequence), wherein glycosylation of the protein may differ due to differences in its preparation.

In one example, a first aminated glycan, provided upon reaction of a first glycan preparation obtained by one production method with a first reducing agent and a first amine, is combined in known or unknown portions with a second aminated glycan, provided upon reaction of a second glycan preparation obtained by a different production method with a second reducing agent and an second amine. In this example, the first reducing agent is isotopically-labeled, and transfers an isotopic atom to the first aminated glycan, while the second reducing agent may or may not be isotopically labeled, and thus may or may not transfer an isotopic atom to the second aminated glycan. Furthermore, the first and second amine may or may not have the same molecular structure, and may or may not be isotopically labeled. The ultimate result of these various combinations is such that the mass of the first aminated glycan is different from that of the second aminated glycan. In such a way, two or more different glycan preparations may be compared using mass spectroscopy.

It is to be understood that when a comparison between two different glycan preparations is being performed, the corresponding aminated glycan preparations can be combined in known or unknown proportions.

In certain embodiments, first and second aminated glycan preparations can be combined in known proportions. If the original glycan preparations include the same glycan species then the mixture of aminated glycan preparations will include first and second aminated versions of this species. As discussed above, these two aminated versions will differ in mass and will therefore generate a pair of peaks in a mass spectrum that are separated by a known mass difference. Quantification of the relative amounts of the common glycan species in the original glycan preparations may then be calculated based on the ratio of the relative intensities of these peaks and the known proportion of the first and second aminated glycan preparations that were combined.

In other embodiments, the first and second aminated glycan preparations can be combined in unknown proportions. A direct comparison between the intensities of pairs of peaks corresponding to the same species will no longer provide a measure of the relative amounts of this species that were present in the original glycan preparations. Instead, one can compare the relative intensities of pairs of peaks that correspond to different species. For example, if two glycan preparations are identical in composition (e.g., they include a set of species at the same concentration) then they should generate pairs of peaks for the different species that have the same relative intensities (e.g., all have a 2:1 ratio). However, if one particular species happens to be present at a different concentration in the two glycan preparations then the relative intensity of the two peaks for that particular species will deviate from those of other species (e.g., it will have a 4:1 ratio if it is enriched in the first glycan preparation or a 1:1 ratio if it is enriched in the second glycan preparation). In general, this approach relies on the fact that variations in the ratio between peaks from the two aminated glycan preparations indicate a difference in the relative amount of a glycan species present (or not present) in the first and second glycan preparations. In certain embodiments, it may prove advantageous to compare the ratio for a glycan species with the ratio for a glycan standard that was added in known amounts (e.g., equal amounts) to the two glycan preparation prior to reductive amination. In order to avoid interfering with the quantification process, the glycan standard will generally comprise a glycan species that is not normally present in the first and second glycan preparations (e.g., without limitation a glycan species that is known to be absent from the glycan preparation of interest, a glycan species that includes a non-naturally occurring modification or sugar, etc.).

Thus, in one aspect, the present disclosure provides a quantification method for determining the relative amount of a glycan species in at least two different glycan preparations, comprising steps of: (i) reacting a first glycan preparation with a first amine and a first reducing agent to provide a first aminated glycan preparation; (ii) reacting a second glycan preparation with a second amine and a second reducing agent to provide a second aminated glycan preparation; (iii) combining an amount of the first aminated glycan preparation with an amount of the second aminated glycan preparation to provide a glycan mixture; (iv) analyzing the glycan mixture by mass spectrometry to provide a mass spectrum which includes at least one pair of peaks that corresponds to aminated versions of a glycan species from the first and second aminated glycan preparations; and (v) determining the relative intensities of the peaks in the at least one pair of peaks; and (vi) quantifying the relative amounts of the glycan species provided in the first and second glycan preparations in light of the relative intensities of the peaks.

In general it is to be understood that the mass of the aminated versions of the same glycan species may differ by any amount. Typically, the mass will differ by between 1 and 10 Daltons; however, in certain embodiments mass differences of more than 10 Daltons may be used. Small mass differences may impair the resolution of peaks from the same glycan species. Large mass differences may cause the peaks from unrelated glycan species to overlap. Thus, in one set of embodiments, the mass may differ by between 1 and 6 Daltons, for example between 2 and 5 Daltons. In one embodiment the mass may differ by 2 Daltons. In one embodiment the mass may differ by 3 Daltons. In one embodiment the mass may differ by 4 Daltons. In another embodiment the mass may differ by 5 Daltons.

The mass difference may be achieved by using the different isotopic analogs of the same molecule. For example, in certain embodiments, the first amine and second amine may have the same chemical structure but include different isotopic labels. For example, in certain embodiments, the first amine is a deuterated (²H) analog of the second amine. In certain embodiments, the first amine is a tritiated (³H) analog of the second amine. In certain embodiments, the first amine is a ¹³C analog of the second amine. In certain embodiments, the first amine is an ¹⁵N analog of the second amine. In certain embodiments, the first amine is an ¹⁸O analog of the second amine. In certain embodiments, the first amine is a ³³S analog of the second amine. In certain embodiments, the first amine is a ³⁴S analog of the second amine. In certain embodiments, the first amine is a ³²P analog of the second amine. In certain embodiments, the first amine is a ²⁹Si analog of the second amine. In certain embodiments, the first amine is a ³⁰Si analog of the second amine. In certain embodiments, the first amine is a ¹⁰B analog of the second amine. In certain embodiments, the first amine is a ¹¹B analog of the second amine.

However, it is to be understood that the methods are not limited to using isotopic analogs and that the same mass difference can also be achieved by using molecules with different chemical structures (e.g., two different functional tags that differ in mass by between 1 and 10 Daltons).

Any mass spectroscopic technique may be used to analyze a glycan mixture according to the methods described herein. As is well known in the art, mass spectroscopy is a method in which charged molecules are accelerated in a vacuum through a magnetic field and then sorted on the basis of mass-to-charge ratio. The ion source is the part of the mass spectrometer that ionizes the molecules under analysis. The ions are then transported by magnetic or electric fields to the mass analyzer. A variety of techniques have been developed for ionizing molecules in a mass spectrometer. Such techniques include, but are not limited to, electrospray ionization (ESI), matrix-assisted laser desorption/ionization (MALDI), fast atom bombardment (FAB), and fourier transform ion cyclotron resonance (FT). Typically, the molecules are charged by bombardment with an adjustable electron beam. Depending on the energy of the electron beam, the molecules may fragment. The process of fragmentation follows simple and predictable chemical pathways and the ions which are formed will reflect the most stable ions and radical ions which that molecule can form. Since the bulk of the ions produced in the mass spectrometer carry a single unit of charge, the value m/z is typically equivalent to the molecular weight of the molecule or fragment. The output of the mass spectrometer shows a plot of relative intensity vs the mass-to-charge ratio (m/z). The most intense peak in the spectrum is termed the base peak and all others are reported relative to its intensity.

Mass spectroscopy may also be combined with one or more separation methods, such as liquid chromatography (LC) or gas chromatography (GC). In liquid chromatography mass spectrometry (LC/MS or LC-MS), compounds are separated chromatographically before they are introduced to the ion source and mass spectrometer. LC-MS differs from GC-MS in that the mobile phase is a liquid rather than a gas.

Exemplary mass spectroscopy methods include one or more tandem ionization and/or separation techniques, or combinations thereof, such as tandem mass spectrometry (MS/MS), electrospray ionization mass spectrometry (ESI-MS), electrospray ionization mass spectrometry/mass spectrometry (ESI-MS/MS), matrix-assisted laser desorption/ionization mass spectrometry (MALDI-MS), matrix-assisted laser desorption/ionization time-of-flight mass spectrometry (MALDI-TOF-MS), fast atom bombardment mass spectrometry (FAB-MS), liquid chromatography mass spectrometry (LC-MS), liquid chromatography mass spectrometry-mass spectrometry (LC-MS/MS), fourier transform ion cyclotron resonance mass spectrometry (FT-MS), and matrix-assisted laser desorption/ionization-time-of-flight/time-of-flight mass spectrometry (MALDI-TOF/TOF-MS).

Tandem mass spectrometry, or MS/MS, involves multiple steps of mass selection or analysis, usually separated by some form of fragmentation. A tandem mass spectrometer is one capable of multiple rounds of mass spectrometry. For example, one mass analyzer can isolate one molecule from many entering a mass spectrometer. A second mass analyzer then stabilizes the molecule ions while they collide with a gas, causing them to fragment by collision-induced dissociation (CID). A third mass analyzer then catalogs the fragments produced from the original molecule. Tandem MS can also be done in a single mass analyzer over time such as in a quadrupole ion trap. There are various methods for fragmenting molecules for tandem MS, including collision-induced dissociation (CID), electron capture dissociation (ECD), electron transfer dissociation (ETD), infrared multiphoton dissociation (IRMPD) and blackbody infrared radiative dissociation (BIRD).

In ESI, the sample solution is sprayed in a fine mist of charged droplets containing sample ions by application of a large negative or positive voltage (typically ±4.5 to ±5 kV). A flow of nitrogen drying gas is directed at droplets and individual positive or negative ions are produced. ESI accommodates a liquid flow of 1 mL/min to 1 mL/min. This ionization technique is particularly suitable for the analysis of polar, thermally labile molecules such as glycans.

MALDI is a laser-based soft ionization technique particularly suitable for the analysis of high molecular weight compounds, including glycans with relative masses up to several hundred kilodalton. For a typical MALDI analysis, the sample and matrix solutions (e.g., 2,5-dihydroxybenzoic acid (DHB), 3,5-dimethoxy-4-hydroxycinnamic acid (sinapinic acid), a-cyano-4-hydroxycinnamic acid (CHCA)), may be premixed or applied directly to the sample support (i.e., sample plate), allowed to dry and then ionized.

TOF is an analysis method used in conjunction with an ionization method which uses an electric field to accelerate the ions through the same potential, and then measures the time they take to reach the detector. If the particles all have the same charge, then their kinetic energies will be identical, and their velocities will depend only on their masses. Lighter ions reach the detector first.

FAB refers to a “soft” ionization technique used in mass spectrometry in which a molecule and a non-volatile chemical protection environment (liquid matrix such as glycerol or 3-nitrobenzyl alcohol) mixture are bombarded by a ˜8 KeV particle beam of usually inert gas such as argon or xenon. Polar molecules, such as glycans may be analyzed by this method.

FT is another analysis method used in conjunction with an ionization method wherein ions, passing through a series of pumping stages at increasingly high vacuum, travel through magnetic field, and subsequently are bent into a circular motion in a plane perpendicular to the field by the Lorentz Force. The frequency of rotation of the ions is dependent on their m/z ratio. Excitation of each individual m/z is achieved by a swept RF pulse across the excitation plates of the cell. When the RF goes off resonance for that particular m/z value, the ions drop back down to their natural orbit (relax), resulting in an FID signal. Deconvolution of this signal by FT methods results in the deconvoluted frequency vs. intensity spectrum which is then converted to the mass vs. intensity spectrum (the mass spectrum). Due to the ion-trap nature of FT-MS, it is possible to measure the ions without destroying them, this enables further experiments to performed on the ions.

In certain embodiments, the glycan mixture is analyzed by a method selected from the group consisting of electrospray ionization mass spectrometry (ESI-MS), electrospray ionization mass spectrometry/mass spectrometry (ESI-MS/MS), matrix-assisted laser desorption/ionization mass spectrometry (MALDI-MS), matrix-assisted laser desorption/ionization time-of-flight mass spectrometry (MALDI-TOF-MS), fast atom bombardment mass spectrometry (FAB-MS), tandem mass spectrometry (MS/MS), liquid chromatography mass spectrometry (LC-MS), liquid chromatography mass spectrometry-mass spectrometry (LC-MS/MS), fourier transform ion cyclotron resonance mass spectrometry (FT-MS), and matrix-assisted laser desorption/ionization-time-of-flight/time-of-flight mass spectrometry (MALDI-TOF/TOF-MS).

A mass spectrum of the glycan mixture will include pairs of peaks that correspond to different aminated versions of the same glycan species, separated by their respective mass difference.

The relative amounts of a given glycan species provided by the first and second glycan preparations can be readily determined based on the relative intensities of the peaks in each pair. In one embodiment this can involve comparing the intensities of the peaks in a pair. In another embodiment this can involve comparing the areas of the peaks. This might require deconvoluting overlapping peaks, e.g., when the mass difference between the different aminated versions of the same glycan species is smaller than the width of the peaks.

It will be appreciated that the labeled glycans may be further analyzed by any technique. For example, the labeled glycans may be analyzed by chromatographic methods, electrophoretic methods, nuclear magnetic resonance (NMR) methods, and combinations thereof.

In some embodiments, the labeled glycans can be analyzed by chromatographic methods, including but not limited to, liquid chromatography (LC), high performance liquid chromatography (HPLC), ultra performance liquid chromatography (UPLC), thin layer chromatography (TLC), amide column chromatography, and combinations thereof.

In some embodiments, the labeled glycans can be analyzed by electrophoretic methods, including but not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof.

In some embodiments, the labeled glycans can be analyzed by nuclear magnetic resonance (NMR) and related methods, including but not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

In some embodiments, the methods described herein allow for detection of glycans that are present at low levels within a population of glycans. For example, the present methods allow for detection of glycan species that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

In some embodiments, the methods described herein allow for detection of particular linkages that are present at low levels within a population of glycans. For example, the present methods allow for detection of particular linkages that are present at levels less than 10%, less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.025%, or less than 0.01% within a population of glycans.

### Glycan Preparations

In one aspect, the methods provide tools for comparing glycan preparations obtained from different sources. The methods described herein are not limited to any specific source or type of glycan preparation.

In one embodiment, the glycan preparations may be obtained from glycoprotein preparations. The present disclosure also contemplates obtaining glycan preparations from glycoprotein preparations which were obtained from cell culture. Methods of providing a glycan preparation from a glycoprotein preparation, and a glycoprotein preparation from a cell, are described below and herein. Different glycoprotein preparations may be obtained by using, for example, different cell types to generate the glycoprotein preparation; by varying cell culture conditions; or by obtaining different glycoprotein samples of the same cell culture at different time increments. As well, different glycan preparations are provided upon varying any one of a number of cleavage, isolation, and/or purification conditions. These and other condition variables are also described below and herein.

In certain embodiments, the first glycan preparation is obtained from a first glycoprotein preparation. In certain embodiments, the second glycan preparation is obtained from a second glycoprotein preparation. In certain embodiments, the first glycoprotein preparation is different from the second glycoprotein preparation.

In certain embodiments, the first glycoprotein preparation is isolated from a cell. In certain embodiments, the first glycoprotein preparation is isolated from a cell's surface. In certain embodiments, the first glycoprotein preparation is isolated from a cell's secretion. In certain embodiments, the first glycoprotein preparation is isolated from a lysed cell.

In certain embodiments, the second glycoprotein preparation is isolated from a cell. In certain embodiments, the second glycoprotein preparation is isolated from a cell's surface. In certain embodiments, the second glycoprotein preparation is isolated from a cell's secretion. In certain embodiments, the second glycoprotein preparation is isolated from a lysed cell.

In certain embodiments, the first and second glycoprotein preparations are obtained from the same cell. In certain embodiments, the first and second glycoprotein preparations are obtained from different cells.

In certain embodiments, the first and second glycoprotein preparations are obtained using the same cell culture conditions. In certain embodiments, the first and second glycoprotein preparations are obtained using different cell culture conditions.

In certain embodiments, the first and second glycoprotein preparations are obtained from the same cell culture at different time increments. In certain embodiments, the first and second glycoprotein preparations are obtained from different cell cultures at the same time increments.

In certain embodiments, the first and second glycoprotein preparations are obtained using the same cleavage conditions. In certain embodiments, the first and second glycoprotein preparations are obtained using different cleavage conditions.

In certain embodiments, the first and second glycoprotein preparations are obtained using the same isolation conditions. In certain embodiments, the first and second glycoprotein preparations are obtained using different isolation conditions.

In certain embodiments, the first and second glycoprotein preparations are obtained using the same purification conditions. In certain embodiments, the first and second glycoprotein preparations are obtained using different purification conditions.

### Growth of Cells in Cell Culture

Any of a variety of cells and/or cell lines capable of protein expression, including for example expression of a therapeutic glycoprotein, can be used to prepare a glycoprotein preparation in accordance with the present disclosure. Any cell that glycosylates at least some of its proteins can be utilized and grown under any conditions that allow such glycosylation to occur. Suitable cells include, but are not limited to, mammalian cells, avian cells, fish cells, insect cells, plant cells, fungal cells, bacterial cells, and hybrid cells. In some embodiments, the cells have been engineered (e.g., genetically and/or chemically) to have one or more glycosylation characteristics more similar to human cells.

Exemplary mammalian cell lines that can be used in accordance with the present disclosure include, but are not limited to, Chinese hamster ovary (CHO) cells, HeLa cells, Madin-Darby canine kidney (MDCK) cells, baby hamster kidney (BHK cells), NSO cells, MCF-7 cells, MDA-MB-438 cells, U87 cells, A172 cells, HL60 cells, A549 cells, SP10 cells, DOX cells, DG44 cells, HEK 293 cells, SHSY5Y, Jurkat cells, BCP-1 cells, COS cells, Vero cells, GH3 cells, 9L cells, 3T3 cells, MC3T3 cells, C3H-10T1/2 cells, NIH-3T3 cells, and C6/36 cells.

Exemplary fish cell lines that can be used in accordance with the present disclosure include, but are not limited to, ZF4 cells, AB9 cells, GAKS cells, OLF-136 cells, CAEP cells, CAF cells, OLHE-131 cells, OLME-104 cells, ULF-23 cells, BRF41 cells, Hepa-E1 cells, Hepa-T1 cells, GEM-81 cells, GEM-199 cells, GEM-218 cells, GAKS cells, D-11 cells, R1 cells, RTG-2 cells, RTO cells, and TPS cells. A more complete list can be found in Fryer and Lannan, 2005, “Three decades of fish cell culture: a current listing of cell lines derived from fishes,” J. Tissue Culture Methods, 16:87-94.

Exemplary insect cell lines that can be used in accordance with the present disclosure include, but are not limited to, SFM cells, Sf21 cells, Sf9 cells, Schneider cells, S2 cells, T.ni cells, SES-MaBr-1 cells, SES-MaBr-3 cells, NIAS-MB-25 cells, NIAS-MaBr-92 cells, FRI-SpIm-1229 cells, SES-MaBr-4 cells, NIAS-LeSe-11 cells, TUAT-SpLi-221 cells, NIAS-PX-64 cells, NIAS-MB-32 cells, NIAS-MaBr-93 cells, SES-MaBr-5 cells, BM-N cells, NIAS-PX-58 cells, MBHL-2 cells, and MBHL-3 cells.

Those of ordinary skill in the art will recognize that this is an exemplary, not a comprehensive, listing of various cell lines that may be used in accordance with the present disclosure. Other cell lines may be advantageously utilized to produce a glycoprotein preparation.

Any of a variety of cell culture media, including complex media and/or serum-free culture media, that are capable of supporting growth of the one or more cell types or cell lines may be used in accordance with the present disclosure. Typically, a cell culture medium contains a buffer, salts, energy source, amino acids (e.g., natural amino acids, non-natural amino acids, etc.), vitamins and/or trace elements. Cell culture media may optionally contain a variety of other ingredients, including but not limited to, carbon sources (e.g., natural sugars, non-natural sugars, etc.), cofactors, lipids, sugars, nucleosides, animal-derived components, hydrolysates, hormones/growth factors, surfactants, indicators, minerals, activators/inhibitors of specific enzymes, and organics (e.g., butyrate, which induces apoptosis, which releases glycosylases, often slows down growth rate of cell, which changes glycosyltransferase levels, which can result in more mature glycosylation; and results in change in energy of cell; chloroquin, which affects intracellular pH; betaine, an osmoprotectant; ammonia, which alters intracellular pH levels and which can change glycosyl transferase efficiency; etc.), and/or small molecule metabolites (e.g., CMP-sialic acid, glucosamine, Bertozzi compounds, etc.). Cell culture media suitable for use in accordance with the present disclosure are commercially available from a variety of sources, e.g., ATCC (Manassas, Va.).

In certain embodiments, one or more of the following media are used to grow cells: RPMI-1640 Medium, Dulbecco's Modified Eagle's Medium, Minimum Essential Medium Eagle, F-12K Medium, Iscove's Modified Dulbecco's Medium. As will be understood by those of ordinary skll in the art, when defined medium that is serum-free and/or peptone-free is used, the medium is typically highly enriched for amino acids and trace elements (see, for example, U.S. Pat. No. 5,122,469 to Mather et al., and U.S. Pat. No. 5,633,162 to Keen et al.). Different cell culture media may affect the glycosylation pattern of glycoproteins expressed in that medium. For example, a given cell culture media may result in production of glycoproteins with an increased glycosylation pattern, a decreased glycosylation pattern, or a combination of increased and decreased glycosylation patterns. One of ordinary skill in the art will be aware of and will be able to choose one or more suitable cell culture media for use in growing cells whose cell surface protein-linked glycans are to be analyzed using certain methods of the present disclosure.

In some embodiments, cells are cultured in batch culture, fed batch culture, perfusion culture, static suspension (e.g., roller bottles, T flasks, microcarriers, T150, etc.), and/or on shakers.

Cells used to generate a glycoprotein preparation can be grown under any of a variety of cell culture conditions. In some embodiments, cells are cultured under cell culture conditions such that a given glycoprotein in a glycoprotein preparation exhibits a desired glycosylation pattern. In some embodiments, one or more cell culture conditions are controlled and/or modified in order to produce glycoproteins that exhibit more desirable glycosylation patterns. Such cell culture conditions include, but are not limited to, pH, CO₂ levels, oxygen levels, culture agitation rate, redox conditions, culture temperature, cell density, density of seed culture, duration of culture, reactor design, sparge rate, and/or osmolarity. One of ordinary skill in the art will be aware of and will be able to control and/or modify one or more cell culture conditions in order to produce glycoproteins that exhibit more desirable glycosylation patterns. As will be recognized by those of ordinary skill in the art, however, it will often be useful to predict and/or verify that a produced glycoprotein exhibits a desirable glycosylation pattern. As such, certain methods comprising the determining overall the glycosylation status of a cell by characterizing the glycosylation pattern of cell surface glycoproteins are useful since such methods permit better prediction and/or control of the glycosylation pattern of a produced therapeutic glycoprotein.

Any of a variety of methods can be used to purify cells from the cell culture medium. In certain embodiments, cells are grown in a suspension culture. In such embodiments, cells may be purified from the cell culture medium by one or more cycles of centrifugation and washing (e.g., with a physiological suitable washing solutions such as phosphate-buffered saline). Care should be taken not to centrifuge the cells with too much force in order to avoid unnecessary cell breakage.

In certain embodiments, cells are grown in an adhesion culture. In such embodiments, cells may be purified from the cell culture medium by first releasing them from the culture surface. For example, cells may be released from the culture surface by subjecting them to EDTA. Those of ordinary skill in the art will be aware of other suitable agents that can be used to released adherent cells from the culture surface. After release, cells may be purified by one or more cycles of centrifugation and washing (e.g., with a physiological suitable washing solutions such as phosphate-buffered saline). As with cells grown in suspension culture, care should be taken not to centrifuge the cells with too much force in order to avoid unnecessary cell breakage.

### Isolation of Cell Surface Glycoproteins

In certain embodiments, one step involved in analyzing cell-surface glycans is liberating such glycans from the surface of the cell. Among the several advantages offered by such embodiments is the fact that a highly pure population of cell-surface glycoproteins can be obtained without significant contamination by glycoproteins that are primarily found inside the cell. For example, using certain methods, lysis of cells is substantially avoided when cell-surface glycoproteins are liberated from the cell. Additionally or alternatively, certain methods disclosed herein offer significant reductions in the number and/or difficulty of manipulation steps as compared to currently available methods.

In certain embodiments, cell-surface glycoproteins are liberated from the cell surface by subjecting the cell to one or more proteases. Proteases cleave amide bonds within a polypeptide chain. Several classes of proteases exist including both chemical and enzymatic agents. Proteolytic enzymes include, for example, serine proteases, threonine proteases, cysteine proteases, aspartic acid proteases, metalloproteases, and glutamic acid proteases. Non-limiting examples of specific proteolytic enzymes that can be used in accordance with the present disclosure include trypsin, chymotrypsin, elastase, subtilisin, proteinase K, pepsin, ficin, bromelin, plasmepsin, renin, chymosin, papain, a cathepsin (e.g. cathepsin K), a caspase (e.g. CASP3, CASP6, CASP7, CASP14), calpain 1, calpain 2, hermolysin, carboxypeptidase A or B, matrix metalloproteinase, a glutamic acid protease, and/or combinations thereof. Those of ordinary skill in the art will be aware of a number of other proteases that can be used in accordance with the present disclosure to release a glycoprotein from the surface of a cell.

Current methods of analyzing cellular glycoproteins, even when explicitly stated to be targeting cell surface glycans, are typically not particularly selective for cell surface glycans. For example, current methods typically employ one or more harsh detergents to extract membrane proteins, after which free sugars are dialyzed away before treatment with agents that remove glycan structures from proteins or polypeptides. Under such conditions, glycan preparations are contaminated by intracellular glycans, e.g., from the endoplasmic reticulum and/or Golgi apparatus. Such intracellular glycans are typically immature, high-mannose glycans. Thus, their inclusion can skew the analysis of glycan structures associated with cell surface glycoproteins.

In some embodiments, analysis of cell surface glycans involves use of detergents to release cell surface glycoproteins from membranes. In some embodiments, however, detergent treatment is minimized or avoided altogether in favor of strategies that minimize disruption of cell membranes. For example, in some embodiments, at least 80%, 85%, 90%, 95%, 96%, 97%, 98%, 99%, or more of the cell membranes remain intact (e.g., as monitored by tryptan blue exclusion). Such methods are advantageous, among other things, because the can reduce or eliminate contamination from immature, high-mannose glycoproteins that are present inside the cell.

In certain embodiments, glycans (in the form of glycopeptides) are liberated from a cell surface by subjecting the cell to one or more proteases. In certain embodiments, cells are subjected to one or more proteases under conditions that minimize disruption of the cell membrane. In some embodiments of the disclosure, glycans are liberated from a cell surface by subjecting the cell to one or more proteases for a limited period of time in order to avoid substantial lysis of the cell membrane. In certain embodiments, a cell is subjected to one or more proteases for a sufficiently limited time such that substantial lysis of the cell membrane does not occur.

For example, a cell may be subjected to one or more proteases for a period of time that is less than about 15, 14, 13, 12, 11, 10, 9, 8, 7, 6, 5, 4, 3, 2, or 1 minute. In certain embodiments, a cell is subjected to one or more proteases for a period of time that is more than 15 minutes so long as substantial lysis of the cell membrane does not occur. For example, a sufficiently low concentration of protease(s), a sufficiently low temperature and/or any of a variety of other factors or conditions may be employed such that the overall protease activity is decreased to a point where substantial lysis of the cell membrane does not occur. Those of ordinary skill in the art will be aware of and will be able to employ factors or conditions that ensure that substantial lysis of the cell membrane does not occur.

In certain embodiments, at least about 50%, 60%, 70%, 80%, 90%, 91%, 92%, 93%, 94%, 95%, 96%, 97%, 98%, 99% or more of cell surface glycans are released from cells, for example by treatment with a protease. To give but one specific example, the present disclosure demonstrated, for instance, that cleavage with trypsin for 15 min at 37° C. results in release of greater than 50% of the cell surface glycans

In certain embodiments, cell surface glycans are liberated by subjecting a cell to one or more proteases (e.g., proteolytic enzymes) at a concentration of at least about 0.1 mg/mL. In certain embodiments, cell surface glycans are liberated by subjecting a cell to one or more proteases (e.g., proteolytic enzymes) at a concentration of less than about 2.0 mg/mL. In certain embodiments, cell surface glycans are liberated by subjecting a cell to one or more proteases (e.g., proteolytic enzymes) at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 5.0, 6.0, 7.0, 8.0, 9.0, 10.0 mg/mL or higher.

In certain embodiments, cell surface glycans are liberated by subjecting a cell to a plurality of proteases. For example, a cell may be subjected to 2, 3, 4, 5, 6, 7, 8, 9, 10, or more proteases to liberate cell surface glycans. Such a plurality of proteases may be administered to the cell simultaneously and/or sequentially. In certain embodiments, cell surface glycans are liberated by subjecting a cell to a plurality of proteases simultaneously, after which the liberated glycans (in the form of glycopeptides) are purified away from the cell.

In certain embodiments, cell surface glycans are liberated by subjecting a cell to a first protease (or plurality of first proteases) for a first period of time, after which the cell is subjected to a second protease (or plurality of second proteases) for a second period of time. Prior to treatment with the second protease, the first protease may optionally be removed and/or inactivated. By way of example, the first protease may be inactivated incubating the protease at a temperature for a time sufficient to inactivate it. Additionally or alternatively, the first protease may be inactived by incubating it with an inhibitor that is specific to the protease (e.g. an antibody or other molecule that specifically binds the first protease and inhibits its catalytic activity). Other methods of inactivating the first protease will be known to those of ordinary skill in the art. In the case where the first protease is inactivated by incubating it with a specific inhibitor, it will be appreciated that the presence of the inhibitor should not substantially inhibit the activity of the second protease.

In certain embodiments the protease(s) are removed and/or inactivated prior to release of glycans. By way of example, a protease may be inactivated incubating the protease at a temperature for a time sufficient to inactivate it. Alternatively or additionally a protease may be inactivated by incubating with an inhibitor or antibody or other molecule that specifically binds to the protease and inhibits its catalytic activity.

Cleavage of Glycans from Glycoproteins

In certain embodiments, cell-surface glycans are cleaved prior to being analyzed. For example, in certain embodiments, one or more glycan structures is cleaved from cell surface glycopeptides after the cell surface glycopeptides have been liberated from the cell (e.g., through treatment with proteases). In certain embodiments, one or more glycan structures is cleaved from cell-surface glycoproteins that have not been liberated from the cell.

In certain embodiments, one or more glycan structures is released through the use of an enzyme or plurality of enzymes that recognizes and cleaves the glycan structures. Any of a variety of glycosidic and other enzymes that cleave glycan structures from cell-surface glycoproteins may be used in accordance with the present disclosure. Several examples of such enzymes are reviewed in R. A. O'Neill, Enzymatic release of oligosaccharides from glycoproteins for chromatographic and electrophoretic analysis, J. Chromatogr. A 720, 201-215. 1996; and S. Prime, et al., Oligosaccharide sequencing based on exo-and endo-glycosidase digestion and liquid chromatographic analysis of the products, J. Chromatogr. A 720, 263-274, 1996, each of which is incorporated herein by reference in its entirety. In certain embodiments, the enzyme PNGase F (Peptide N-Glycosidase F) is used to remove glycans from a glycoprotein. PNGase F is an amidase that cleaves the amide bond between the innermost GlcNAc and asparagine residues of high mannose, hybrid, and complex oligosaccharides from N-linked glycoproteins.

To improve the accessibility of the glycosylation site to a cleavage enzyme, most glycoproteins require a protein denaturation step. Typically, this is accomplished by using detergents (e.g., SDS) and/or disulfide-reducing agents (e.g., beta-mercaptoethanol), although methods of denaturing a glycoprotein for use in accordance with the present disclosure are not limited to the use of such agents. For example, exposure to high temperature can be sufficient to denature a glycoprotein such that a suitable enzyme for cleaving glycan structures is able to access the cleavage site. In certain embodiments, a glycoprotein is denatured by incubating the glycoprotein at temperature of 80, 81, 82, 83, 84, 85, 86, 87, 88, 89, 90, 91, 92, 93, 94, 95, 96, 97, 98, 99, 100 degrees Celsius, or higher for a period of time sufficient to denature the glycoprotein.

In certain embodiments, a combination of detergents, disulfide-reducing agents, high temperature, and/or other agents or reaction conditions is employed to denature a glycoprotein. Those of ordinary skill in the art will be aware of suitable conditions, incubation times, etc. that will be sufficient to denature a glycoprotein. It is noted that oligosaccharides located at conserved Fc sites in immunoglobulin G (IgG) are easily cleaved by PNGase F. Thus, a protein denaturation step is typically not required for IgG molecules when this enzyme is used. PNGase F is also capable of removing oligosaccharides in dilute ammonium hydroxide solution, is stable in 2.5M urea at 37 C for 24 h, and still possesses 40% of its activity in 5 M urea. Thus, PNGase F has the advantage that it is capable of cleaving glycans from glycoproteins under certain denaturation conditions.

Other suitable enzymes that can be used to cleave glycan structures from glycoproteins in accordance with the present disclosure include, but are not limited to, PNGase A and Endo-H. Those of ordinary skill in the art will be aware of other suitable enzymes for cleavage of glycans from glycoproteins. In certain embodiments, a plurality of enzymes is used to cleave glycan structures from a glycoprotein. In certain embodiments, such a plurality of cleavage enzymes is administered simultaneously. In certain embodiments, such a plurality of cleavage enzymes is administered sequentially.

In certain embodiments, one or more glycan structures is cleaved from cell-surface glycoproteins through the use of an agent other than an enzyme. In certain embodiments, a chemical agent or plurality of chemical agents can be used to cleave glycan structures from glycoproteins. For example, use of the chemical hydrazine has been successfully employed to cleave glycan structures. As another non-limiting example, it has been suggested that a mixture of ammonia-ammonium carbonate can be used for alkaline release of both the N- and O-linked oligosaccharides in their native form (see Y. Huang, et al., Microscale nonreductive release of O-linked glycans for subsequent analysis through MALDI mass spectrometry and capillary electrophoresis, Anal. Chem. 73, 6063-60, 2001, incorporated herein by reference in its entirety). Those of ordinary skill in the art will be aware of other suitable chemical agents that can be used in accordance with the present disclosure. In some cases, use of a chemical agent to cleave glycan structures from a glycoprotein results in protein degradation as well as cleavage. However, after cleavage, the glycan structure is often purified away from the protein component of the glycoprotein before analysis and/or characterization. In such situations, degradation of the protein component after treatment with a chemical agent is not detrimental to the practice of the methods. In some cases, degradation of the protein component may even aid in the process of purifying the cleaved glycan structure(s).

In some embodiments, glycans that have been released from a glycoprotein and/or released from a cell surface can be digested with one or more exoglycosidases, and the structure and/or composition of the digestion products can be analyzed.

Exoglycosidases are enzymes which cleave terminal glycosidic bonds from the non-reducing end of glycans. They are typically highly specific to particular monosaccharide linkages and anomericity (α/β). In some embodiments, neighboring branching patterns can affect exoglycosidase specificity. Exoglycosidase treatment usually results in glycans of standard antennary linkages being cleaved down to the pentasaccharide core (M3N2) containing 3 mannose and 2 GlcNAc residues. However, unusually-modified species (e.g. antennary fucosylated species, high-mannose and hybrid glycans, lactosamine-extended glycans, sulfated or phosphorylated glycans, etc.) are resistant to exoglycosidase treatment and can be chromatographically resolved and quantified relative to the M3N2 pentasaccharide.

In some embodiments, exoglycosidases used in accordance with the present disclosure recognize and cleave only one particular type of glycosidic linkage. In some embodiments, exoglycosidases used in accordance with the present disclosure recognize and cleave more than one particular type of glycosidic linkage. Exemplary exoglycosidases that can be used in accordance with the present disclosure include, but are not limited to, sialidase, galactosidase, hexosaminidase, fucosidase, and mannosidase. Exoglycosidases can be obtained from any source, including commercial sources (e.g. from QA-Bio, ProZyme, Roche, Sigma, NEB, EMD, Glyko, etc.). Alternatively or additionally, exoglycosidases can be isolated and/or purified from a cellular source (e.g. bacteria, yeast, plant, etc.).

In some embodiments, exoglycosidases (e.g. sialidases, galactosidases, hexosaminidases, fucosidases, and mannosidases) can be divided into multiple categories or “subsets.” In some embodiments, the different subsets display different abilities to cleave different types of linkages. Table 1 presents some exemplary exoglycosidases, their linkage specificities, and the organism from which each is derived. One of ordinary skill in the art will appreciate that this is an exemplary, not a comprehensive, list of exoglycosidases, and that any exoglycosidase having any linkage specificity may be used in accordance with the present disclosure.

TABLE 1
Exoglycosidases

| Enzyme class | EC #* | Activity | Organism |
|---|---|---|---|
| α-Sialidase | 3.2.1.18 | α-2/3,6,8 (usually not linkage- | Arthrobacter ureafaciens |
| | | specific) | Vibrio cholerae |
| | | | Clostridium perfringens |
| | | α-2,3 (NeuAc from | Salmonella typhimurium |
| | | oligosaccharides) | Streptococcus pneumonia |
| | | α-2/3,6 (NeuAc from complex) | Clostridium perfringens |
| β-Galactosidase | 3.2.1.23 | β-1/3,4,6 Gal linkages | Bovine testis |
| | | | Xanthamonas species |
| | | | Streptococcus species |
| | | | E. coli |
| | | β-1/4,6 Gal linkages | Jack bean |
| | | β-1,4 Gal linkage | Streptococcus pneumonia |
| | | β-1,3-Gal linkage | E. coli |
| | | | Xanthomonas species |
| | | β-1/3,6-Gal linkages | Xanthomonas species |
| | | | E. coli |
| β-Hexosaminidase | 3.2.1.52 | β-1/2,3,4,6 hexosamines | Streptococcus plicatus |
| | 3.2.1.30 | | Streptococcus pneumonia |
| | | | Bacteroides |
| | | | Jack bean |
| α-Fucosidase | 3.2.1.51 | α-1-3,4-Fuc (usually de- | Xanthomonas |
| | 3.2.1.111 | glycosylate Lewis structure) | Almond meal |
| | | α-1/2,3,4,6-Fuc (usually has broad | Bovine kidney |
| | | specificity) | C. meningosepticum |
| | | α-1,6-Fuc | E. coli |
| | | α-1,2-Fuc | Xanthomonas |
| α-Mannosidase | 3.2.1.24 | α-1/2,3,6-Man | Jack bean |
| | | α-1/2,3-Man | Xanthomonas manihotis |
| | | α-1,6-Man (typically a core | Xanthomonas species |
| | | mannosidase) | |
| | | α-1,2-Man | Aspergillus saitoi |
| β-Mannosidase | 3.2.1.25 | α-1,4-Man | Helix pomatia |

*“EC #” refers to Enzyme Commission registration number

According to the present disclosure, glycans that have been released from a glycoprotein and/or a cell surface can be digested with any exoglycosidase. In certain embodiments, glycans are digested by subjecting a population of glycans to a plurality of exoglycosidases. For example, a population of glycans may be subjected to 2, 3, 4, 5, 6, 7, 8, 9, 10, or more exoglycosidases. In some embodiments, multiple exoglycosidases are administered simultaneously. In some embodiments, multiple exoglycosidases are administered sequentially. In some embodiments, varying the identity of the exoglycosidases which are administered reveals information about glycan structure and/or composition. In some embodiments, varying the sequence in which multiple exoglycosidases are administered reveals information about glycan structure and/or composition.

In some embodiments, sequential digestion with multiple exoglycosidases reveals information about glycan structure and/or composition that is different from information revealed by simultaneous digestion with the same set of exoglycosidases. In some embodiments, sequential digestion with multiple exoglycosidases reveals information about glycan structure and/or composition that is the same information revealed by simultaneous digestion with the same set of exoglycosidases. For a more complete discussion of the utility of exoglycosidase digestion in the analysis of glycan structure, see co-pending U.S. provisional patent application U.S. Ser. No. 60/923,688, filed Apr. 16, 2007, by Parsons et al., entitled “CHARACTERIZATION OF N-GLYCANS USING EXOGLYCOSIDASES,” which is incorporated herein by reference.

### Applications

It will be appreciated that the techniques described herein can be utilized in any of a variety of applications. In general, these techniques are useful in any application that involves the structural characterization of glycans. Techniques of the present disclosure may be particularly useful in the comparison of glycan species in two or more glycan preparations.

Methods in accordance with the disclosure can be applied to glycans obtained from a wide variety of sources including, but not limited to, therapeutic formulations (e.g., erythropoietin, insulin, human growth hormone, etc.), commercial biological products (e.g., those presented in a table below), and biological samples. A biological sample may undergo one or more analysis and/or purification steps prior to or after being analyzed according to the present disclosure. To give but a few examples, in some embodiments, a biological sample is treated with one or more proteases and/or exoglycosidases (e.g., so that glycans are released); in some embodiments, glycans in a biological sample are labeled with one or more detectable markers or other agents that may facilitate analysis by, for example, mass spectrometry or NMR. Any of a variety of separation and/or isolation steps may be applied to a biological sample in accordance with the present disclosure.

The methods can be utilized to analyze glycans in any of a variety of states including, for instance, free glycans; glycoconjugates (e.g., glycopeptides, glycolipids, proteoglycans, etc.); cell-associated glycans (e.g., nucleus-, cytoplasm-, cell membrane-associated glycans, etc.); glycans associated with cellular, extracellular, intracellular, and/or subcellular components (e.g., proteins); glycans in extracellular space (e.g., cell culture medium) etc.

Methods of the present disclosure may be used in one or more stages of process development for the production of a therapeutic or other commercially relevant glycoprotein of interest. Non-limiting examples of such process development stages that can be employ methods of the present disclosure include cell selection, clonal selection, media optimization, culture conditions, process conditions, and/or purification procedure. Those of ordinary skill in the art will be aware of other process development stages.

The methods can also be utilized to monitor the extent and/or type of glycosylation occurring in a particular cell culture, thereby allowing adjustment or possibly termination of the culture in order, for example, to achieve a particular desired glycosylation pattern or to avoid development of a particular undesired glycosylation pattern.

The methods can also be utilized to assess glycosylation characteristics of cells or cell lines that are being considered for production of a particular desired glycoprotein (for example, even before the cells or cell lines have been engineered to produce the glycoprotein, or to produce the glycoprotein at a commercially relevant level).

In some embodiments of the disclosure, a desired glycosylation pattern for a particular target glycoprotein (e.g., a cell surface glycoprotein) is known, and the technology described herein allows monitoring of culture samples to assess progress of the production along a route known to produce the desired glycosylation pattern. For example, where the target glycoprotein is a therapeutic glycoprotein, for example having undergone regulatory review in one or more countries, it will often be desirable to monitor cultures to assess the likelihood that they will generate a product with a glycosylation pattern as close to the established glycosylation pattern of the pharmaceutical product as possible, whether or not it is being produced by exactly the same route. As used herein, “close” refers to a glycosylation pattern having at least about a 75%, 80%, 85%, 90%, 95%, 98%, or 99% correlation to the established glycosylation pattern of the pharmaceutical product. In such embodiments, samples of the production culture are typically taken at multiple time points and are compared with an established standard or with a control culture in order to assess relative glycosylation.

In some embodiments, a desired glycosylation pattern will be more extensive. For example, in some embodiments, a desired glycosylation pattern shows high (e.g., greater than about 60%, 65%, 70%, 75%, 80%, 85%, 90%, 95% or more) occupancy of glycosylation sites; in some embodiments, a desired glycosylation pattern shows, a high degree of branching (e.g., greater than about 60%, 65%, 70%, 75%, 80%, 85%, 90%, 95% or more have tri or tetraantennary structures).

In some embodiments, a desired glycosylation pattern will be less extensive. For example, in some embodiments, a desired glycosylation pattern shows low (e.g., less than about 35%, 30%, 25%, 20%, 15% or less) occupancy of glycosylation sites; and/or a low degree of branching (e.g., less than about 20%, 15%, 10%, 5%, or less have tri or tetraantennary structures).

In some embodiments, a desired glycosylation pattern will be more extensive in some aspects and less extensive than others. For example, it may be desirable to employ a cell line that tends to produce glycoproteins with long, unbranched oligosaccharide chains. Alternatively, it may be desirable to employ a cell line that tends to produce glycoproteins with short, highly branched oligosaccharide chains.

In some embodiments, a desired glycosylation pattern will be enriched for a particular type of glycan structure. For example, in some embodiments, a desired glycosylation pattern will have low levels (e.g., less than about 20%, 15%, 10%, 5%, or less) of high mannose or hybrid structures, high (e.g., more than about 60%, 65%, 70%, 75%, 80%, 85%, 90% or more) levels of high mannose structures, or high (e.g., more than about 60%, 65%, 70%, 75%, 80%, 85%, 90% or more; for example at least one per glycoprotein) or low (e.g., less than about 20%, 15%, 10%, 5%, or less) levels of phosphorylated high mannose.

In some embodiments, a desired glycosylation pattern will include at least about one sialic acid. In some embodiments, a desired glycosylation pattern will include a high (e.g., greater than about 60%, 65%, 70%, 75%, 80%, 85%, 90%, 95% or more) level of termini that are sialylated. In some embodiments, a desired glycosylation pattern that includes sialylation will show at least about 85%, 90%, 95% or more N-acetylneuraminic acid and/or less than about 15%, 10%, 5% or less N-glycolylneuraminic acid.

In some embodiments, a desired glycosylation pattern shows specificity of branch elongation (e.g., greater than about 50%, 55%, 60%, 65%, 70% or more of extension is on α1,6 mannose branches, or greater than about 50%, 55%, 60%, 65%, 70% or more of extension is on α1,3 mannose branches).

In some embodiments, a desired glycosylation pattern will include a low (e.g., less than about 20%, 15%, 10%, 5%, or less) or high (e.g., more than about 60%, 65%, 70%, 75%, 80%, 85%, 90%, 95%, or more) level of core fucosylation.

Whether or not monitoring production of a particular target protein for quality control purposes, the methods may be utilized, for example, to monitor glycosylation at particular stages of development, or under particular growth conditions.

In some particular embodiments, the methods can be used to characterize and/or control or compare the quality of therapeutic products. To give but one example, methodologies can be used to assess glycosylation in cells producing a therapeutic protein product. Particularly given that glycosylation can often affect the activity, bioavailability, or other characteristics of a therapeutic protein product, methods for assessing cellular glycosylation during production of such a therapeutic protein product are particularly desirable. Among other things, the methods can facilitate real time analysis of glycosylation in production systems for therapeutic proteins.

Representative therapeutic glycoprotein products whose production and/or quality can be monitored in accordance with the methods include, for example, any of a variety of hematologic agents (including, for instance, erythropoietins, blood-clotting factors, etc.), interferons, colony stimulating factors, antibodies, enzymes, and hormones.

Representative commercially available glycoprotein products include, for example:

| Protein Product | Reference Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| laronidase | Aldurazyme ® |
| interferon alfa-N3, human leukocyte | Alferon N ® |
| derived | |
| human antihemophilic factor | Alphanate ® |
| virus-filtered human coagulation factor IX | AlphaNine ® SD |
| Alefacept; recombinant, dimeric fusion | Amevive ® |
| protein LFA3-Ig | |
| bivalirudin | Angiomax ® |
| darbepoetin alfa | Aranesp ™ |
| bevacizumab | Avastin ™ |
| interferon beta-1a; recombinant | Avonex ® |
| coagulation factor IX | BeneFix ™ |
| Interferon beta-1b | Betaseron ® |
| Tositumomab | Bexxar ® |
| antihemophilic factor | Bioclate ™ |
| human growth hormone | BioTropin ™ |
| botulinum toxin type A | Botox ® |
| alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune Fab, ovine | DigiFab ™ |
| rasburicase | Elitek ® |
| etanercept | Enbrel ® |
| epoietin alfa | Epogen ® |
| cetuximab | Erbitux ™ |
| algasidase beta | Fabrazyme ® |
| urofollitropin | Fertinex ™ |
| follitropin beta | Follistim ™ |
| teriparatide | Forteo ® |
| human somatropin | GenoTropin ® |
| glucagon | GlucaGen ® |
| follitropin alfa | Gonal-F ® |
| antihemophilic factor | Helixate ® |
| Antihemophilic Factor; Factor XIII | Hemofil ® |
| insulin | Humalog ® |
| antihemophilic factor/von Willebrand | Humate-P ® |
| factor complex-human | |
| somatotropin | Humatrope ® |
| adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| palifermin | Kepivance |
| anakinra | Kineret ™ |
| antihemophilic factor | Kogenate ® FS |
| insulin glargine | Lantus ® |
| granulocyte macrophage colony- | Leukine ®/Leukine ® Liquid |
| stimulating factor | |
| lutropin alfa, for injection | Luveris |
| OspA lipoprotein | LYMErix ™ |
| ranibizumab | Lucentis ® |
| gemtuzumab ozogamicin | Mylotarg ™ |
| galsulfase | Naglazyme ™ |
| nesiritide | Natrecor ® |
| pegfilgrastim | Neulasta ™ |
| oprelvekin | Neumega ® |
| filgrastim | Neupogen ® |
| fanolesomab | NeutroSpec ™ (formerly LeuTech ®) |
| somatropin [rDNA] | Norditropin ®/Norditropin Nordiflex ® |
| insulin; zinc suspension; | Novolin L ® |
| insulin; isophane suspension | Novolin N ® |
| insulin, regular; | Novolin R ® |
| insulin | Novolin ® |
| coagulation factor VIIa | NovoSeven ® |
| somatropin | Nutropin ® |
| immunoglobulin intravenous | Octagam ® |
| PEG-L-asparaginase | Oncaspar ® |
| abatacept, fully human soluable fusion | Orencia ™ |
| protein | |
| muromomab-CD3 | Orthoclone OKT3 ® |
| human chorionic gonadotropin | Ovidrel ® |
| peginterferon alfa-2a | Pegasys ® |
| pegylated version of interferon alfa-2b | PEG-Intron ™ |
| Abarelix (injectable suspension); | Plenaxis ™ |
| gonadotropin-releasing hormone antagonist | |
| epoietin alfa | Procrit ® |
| aldesleukin | Proleukin, IL-2 ® |
| somatrem | Protropin ® |
| dornase alfa | Pulmozyme ® |
| Efalizumab; selective, reversible T-cell | Raptiva ™ |
| blocker | |
| combination of ribavirin and alpha | Rebetron ™ |
| interferon | |
| Interferon beta 1a | Rebif ® |
| antihemophilic factor | Recombinate ® |
| rAHF/ntihemophilic factor | ReFacto ® |
| lepirudin | Refludan ® |
| infliximab | Remicade ® |
| abciximab | ReoPro ™ |
| reteplase | Retavase ™ |
| rituximab | Rituxan ™ |
| interferon alfa-2a | Roferon-A ® |
| somatropin | Saizen ® |
| synthetic porcine secretin | SecreFlo ™ |
| basiliximab | Simulect ® |
| eculizumab | Soliris ® |
| pegvisomant | Somavert ® |
| Palivizumab; recombinantly produced, | Synagis ™ |
| humanized mAb | |
| thyrotropin alfa | Thyrogen ® |
| tenecteplase | TNKase ™ |
| natalizumab | Tysabri ® |
| human immune globulin intravenous 5% | Venoglobulin-S ® |
| and 10% solutions | |
| interferon alfa-n1, lymphoblastoid | Wellferon ® |
| drotrecogin alfa | Xigris ™ |
| Omalizumab; recombinant DNA-derived | Xolair ® |
| humanized monoclonal antibody targeting | |
| immunoglobulin-E | |
| daclizumab | Zenapax ® |
| ibritumomab tiuxetan | Zevalin ™ |
| Somatotropin | Zorbtive ™ (Serostim ®) |

In some embodiments, the disclosure provides methods in which glycans from different sources or samples are compared with one another. In certain embodiments, the disclosure provides methods used to monitor the extent and/or type of glycosylation occuring in different cell cultures. In some such examples, multiple samples from the same source are obtained over time, so that changes in glycosylation patterns (and particularly in cell surface glycosylation patterns) are monitored. In some embodiments, one of the samples is a historical sample or a record of a historical sample. In some embodiments, one of the samples is a reference sample. For example, in certain embodiments, the disclosure provides methods used to monitor the extent and/or type of glycosylation occuring in different cell cultures.

In some embodiments, glycans from different cell culture samples prepared under conditions that differ in one or more selected parameters (e.g., cell type, culture type [e.g., continuous feed vs batch feed, etc.], culture conditions [e.g., type of media, presence or concentration of particular component of particular medium, osmolarity, pH, temperature, timing or degree of shift in one or more components such as osmolarity, pH, temperature, etc.], culture time, isolation steps, etc.) but are otherwise identical, are compared, so that effects of the selected parameter(s) on glycosylation patterns are determined. In certain embodiments, glycans from different cell culture samples prepared under conditions that differ in a single selected parameter are compared so that effect of the single selected parameter on glycosylation patterns is determined. Among other applications, therefore, use of techniques as described herein may facilitate determination of the effects of particular parameters on glycosylation patterns in cells.

In some embodiments, glycans from different batches of a glycoprotein of interest (e.g., a therapeutic glycoprotein), whether prepared by the same method or by different methods, and whether prepared simultaneously or separately, are compared. In such embodiments, the methods facilitate quality control of glycoprotein preparation. Features of the glycan analysis can be recorded, for example in a quality control record. As indicated above, in some embodiments, a comparison is with a historical record of a prior or standard batch of glycoprotein. In some embodiments, a comparison is with a reference glycoprotein sample.

In some embodiments, glycans from different batches of a glycoprotein of interest (e.g., a therapeutic glycoprotein), whether prepared by the same method or by different methods, and whether prepared simultaneously or separately, are compared. In such embodiments, the methods facilitate quality control of glycoprotein preparation. Alternatively or additionally, some such embodiments facilitate monitoring of progress of a particular culture producing a glycoprotein of interest (e.g., when samples are removed from the culture at different time points and are analyzed and compared to one another). In any of these embodiments, features of the glycan analysis can be recorded, for example in a quality control record. As indicated above, in some embodiments, a comparison is with a historical record of a prior or standard batch and/or with a reference sample of glycoprotein.

In certain embodiments, techniques of the present disclosure are applied to glycans that are present on the surface of cells. In some such embodiments, the analyzed glycans are substantially free of non-cell-surface glycans. In some such embodiments, the analyzed glycans, when present on the cell surface, are present in the context of one or more cell surface glycoconjugates (e.g., glycoproteins or glycolipids).

In some particular embodiments, cell surface glycans are analyzed in order to assess glycosylation of one or more target glycoproteins of interest, particularly where such target glycoproteins are not cell surface glycoproteins. Such embodiments can allow one to monitor glycosylation of a target glycoprotein without isolating the glycoprotein itself. In certain embodiments, the present disclosure provides methods of using cell-surface glycans as a readout of or proxy for glycan structures on an expressed glycoprotein of interest. In certain embodiments, such methods include, but are not limited to, post process, batch, screening or “in line” measurements of product quality. Such methods can provide for an independent measure of the glycosylation pattern of a produced glycoprotein of interest using a byproduct of the production reaction (e.g., the cells) without requiring the use of destruction of any produced glycoprotein. Furthermore, methods of the present disclosure can avoid the effort required for isolation of product and the potential selection of product glycoforms that may occur during isolation.

In certain embodiments, techniques of the present disclosure are applied to glycans that are secreted from cells. In some such embodiments, the analyzed glycans are produced by cells in the context of a glycoconjugate (e.g., a glycoprotein or glycolipid).

Techniques described herein can be used to detect desirable or undesirable glycans, for example to detect or quantify the presence of one or more contaminants in a product, or to detect or quantify the presence of one or more active or desired species.

In various embodiments the methods can be used to detect biomarkers indicative of, e.g., a disease state, prior to the appearance of symptoms and/or progression of the disease state to an untreatable or less treatable condition, by detecting one or more specific glycans whose presence or level (whether absolute or relative) may be correlated with a particular disease state (including susceptibility to a particular disease) and/or the change in the concentration of such glycans over time.

In certain embodiments, the methods facilitate detection of glycans that are present at very low levels in a source (e.g., a biological sample). In such embodiments, it is possible to detect and optionally quantify the levels of glycans comprising between 0.1% and 5%, e.g., between 0.1% and 2%, e.g., between 0.1% and 1% of a glycan preparation. In certain embodiments, it is possible to detect and optionally quantify the levels of glycans at between about 0.1 fmol to about 1 mmol.

In some embodiments, the techniques may be combined with one or more other technologies for the detection, analysis, and or isolation of glycans or glycoconjugates. The methods will be more specifically illustrated with reference to the following examples. However, it should be understood that the methods are not limited by these examples in any manner.

### EXAMPLES

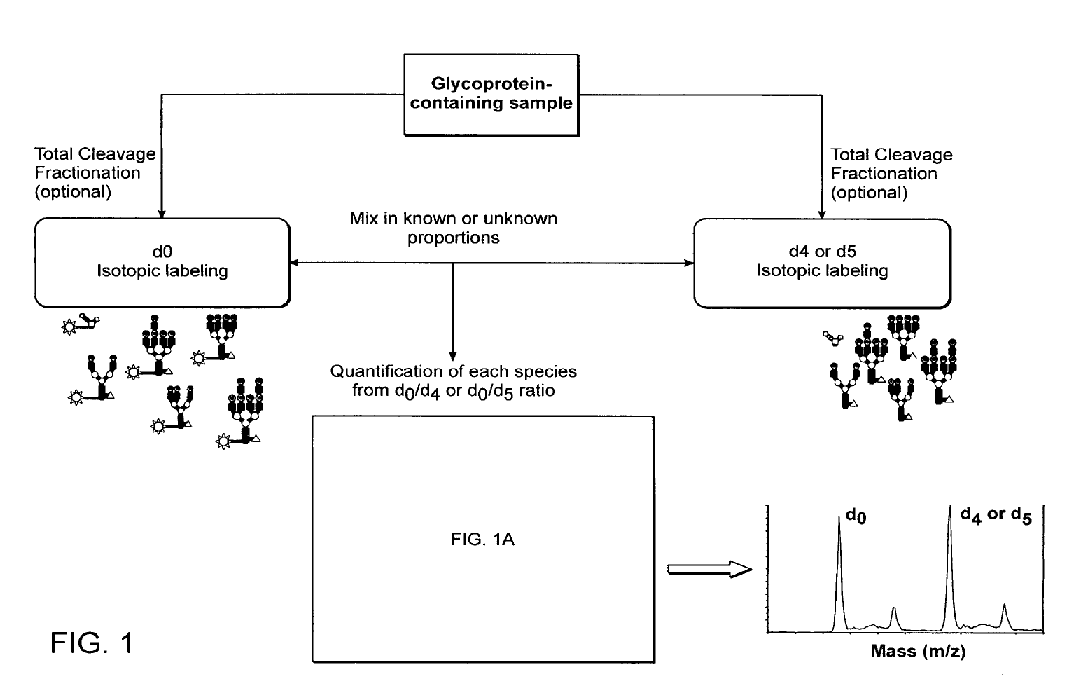

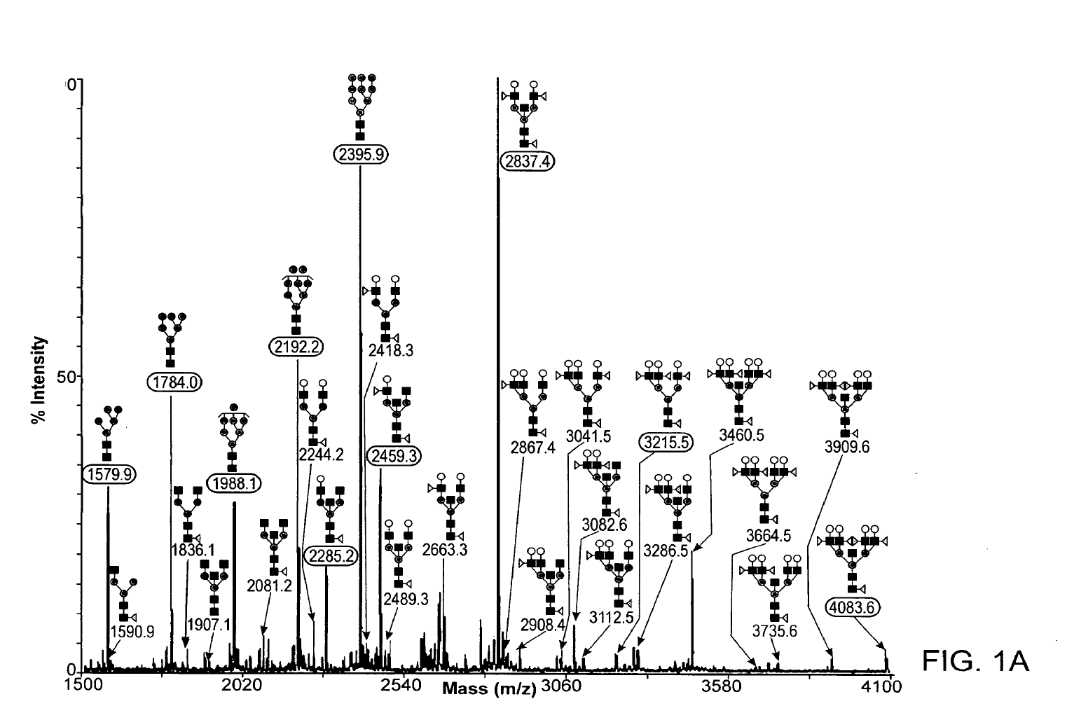

FIG. 1 shows a schematic depicting one embodiment of the comparative quantification of glycans according to the present disclosure. As shown, two samples are taken from a single glycoprotein preparation. Glycans are then cleaved from the glycoproteins using two different methods (e.g., using different proteases and/or glycosidases) to produce two different glycan preparations. The two glycan preparations are then labeled via reductive amination, e.g., with 2-aminobenzamide (2-AB); 2-aminobenzoic acid (2-AA); or 2-aminopyridine (2-AP)) containing different isotopic contents. For example, one glycan preparations is labeled with non-isotopically enriched 2-AB while the other glycan preparation is labeled with isotopically enriched 2-AB (e.g., d₃ or d₄) using an isotopically enriched reducing agent (e.g., d₁) to produce a mass difference (e.g., of 4 or 5 Daltons). After purification, the labeled glycan preparations are mixed in known proportions and analyzed using mass spectroscopy. Relative quantifications of species present in the two glycan preparations are then determined from the ratio of the ion count intensities or areas of the different isotopes of co-eluting species.

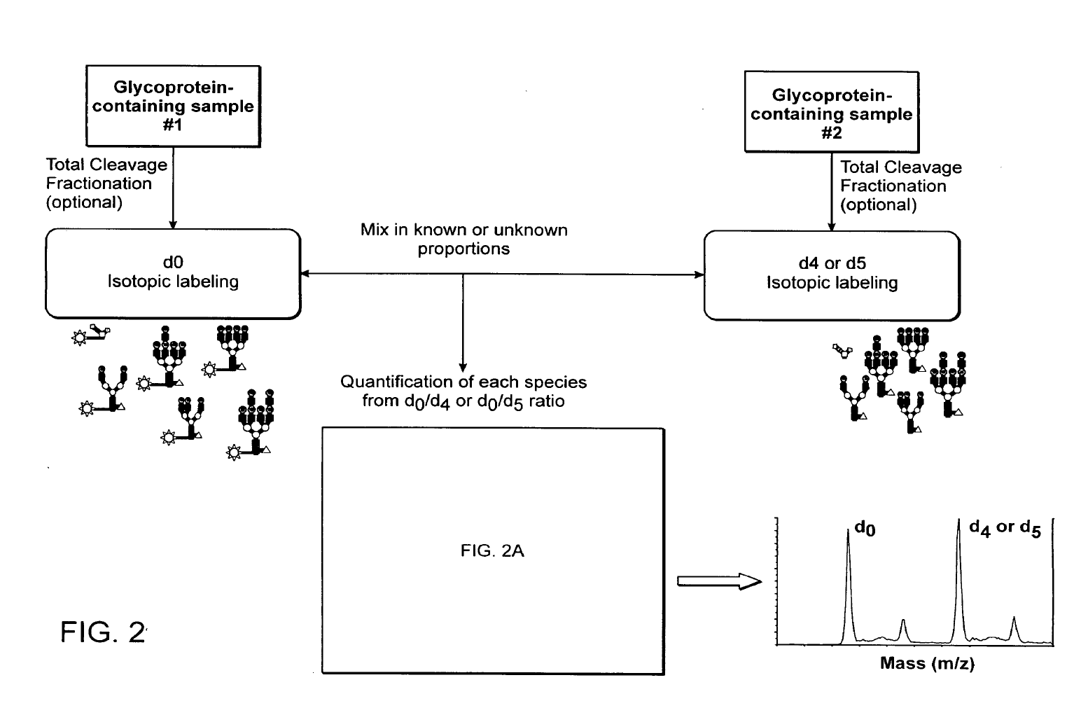

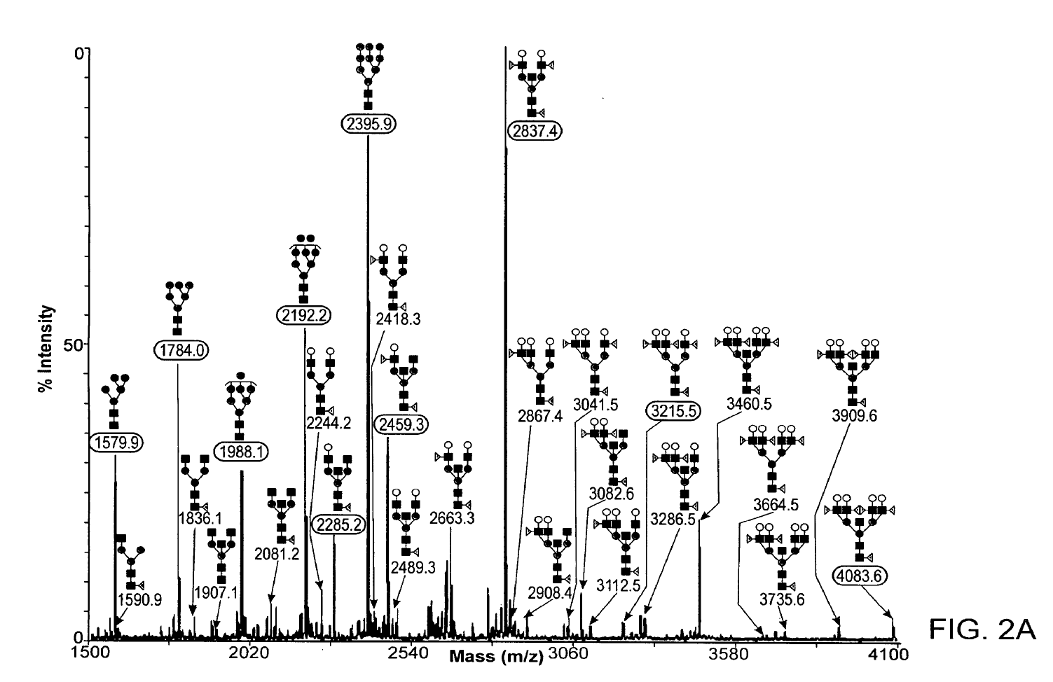

FIG. 2 shows a schematic depicting another embodiment of the comparative quantification of glycans according to the present disclosure. As shown, two samples are taken from different glycoprotein preparations. Glycans are then cleaved from the glycoproteins to produce two different glycan preparations. The two glycan preparations are then labeled via reductive amination, e.g., with 2-aminobenzamide (2-AB); 2-aminobenzoic acid (2-AA); or 2-aminopyridine (2-AP) containing different isotopic contents. For example, one glycan preparation is labeled with non-isotopically enriched 2-AB while the other glycan preparation is labeled with isotopically enriched 2-AB (e.g., d₃) using an isotopically enriched reducing agent (e.g., d₁) to produce a mass difference (e.g., of 4 Daltons). After purification, the labeled glycan preparations are mixed in known proportions and analyzed using mass spectroscopy. Relative quantifications of species present in the two glycan preparations are then determined from the ratio of the ion count intensities of the different isotopes of similar species.

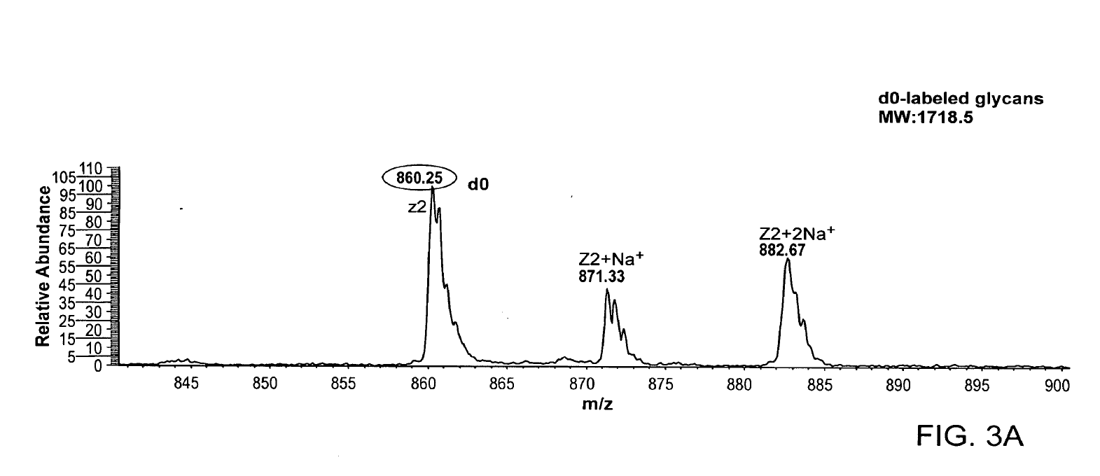

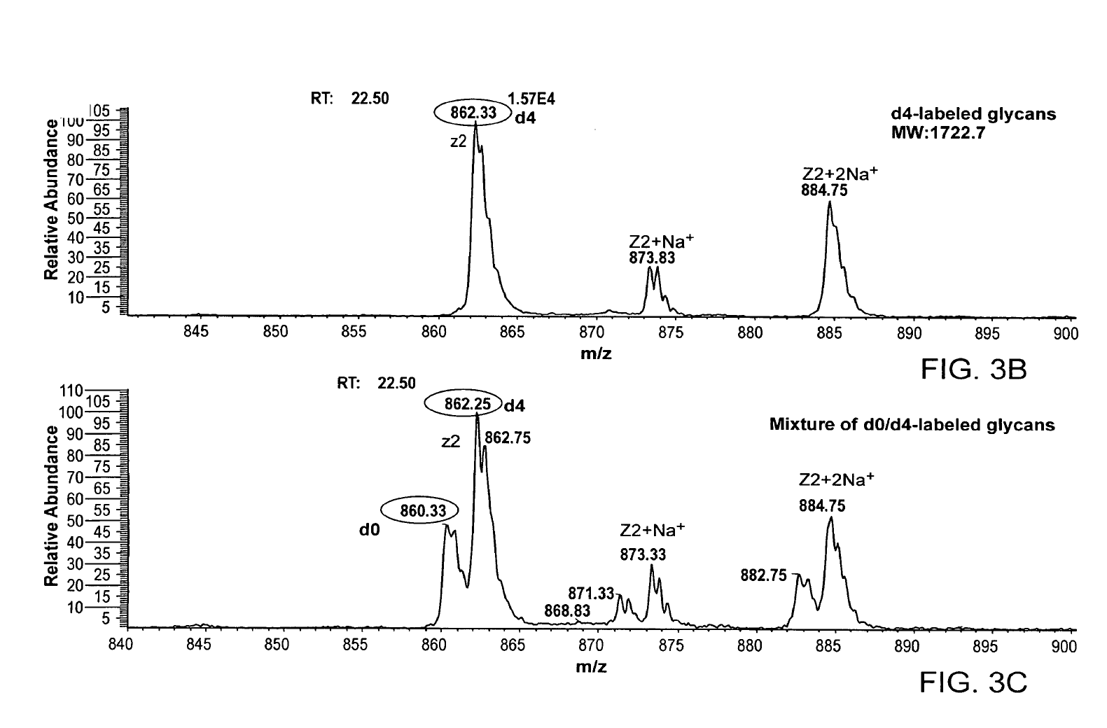

FIG. 3 provides mass spectra that were generated from glycan preparations prepared according to the scheme of FIG. 1. The top mass spectrum corresponds to a glycan preparation that was labeled with non-isotopically enriched 2-AB (d₀). The middle mass spectrum corresponds to a glycan preparation that was labeled with isotopically enriched 2-AB (d3) using an isotopically enriched reducing agent (d₁). The bottom mass spectrum corresponds to a mixture of the two glycan preparations. The quantity of a given glycan species in the two preparations can be quantified by comparing the d₀ and d₄ peaks (e.g., peak intensity or area).

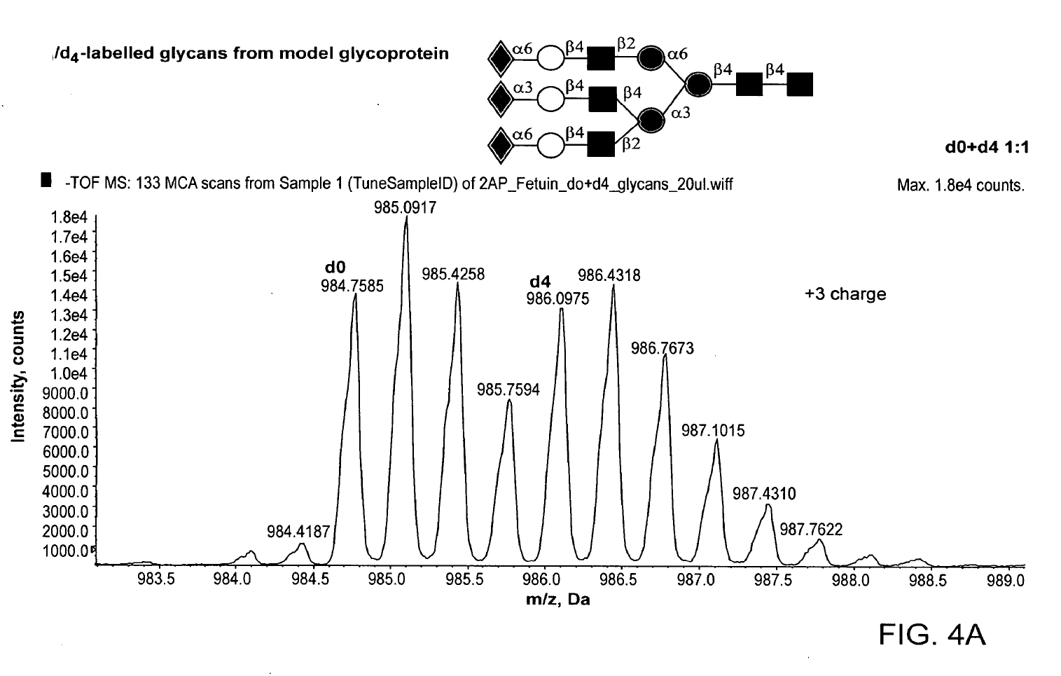

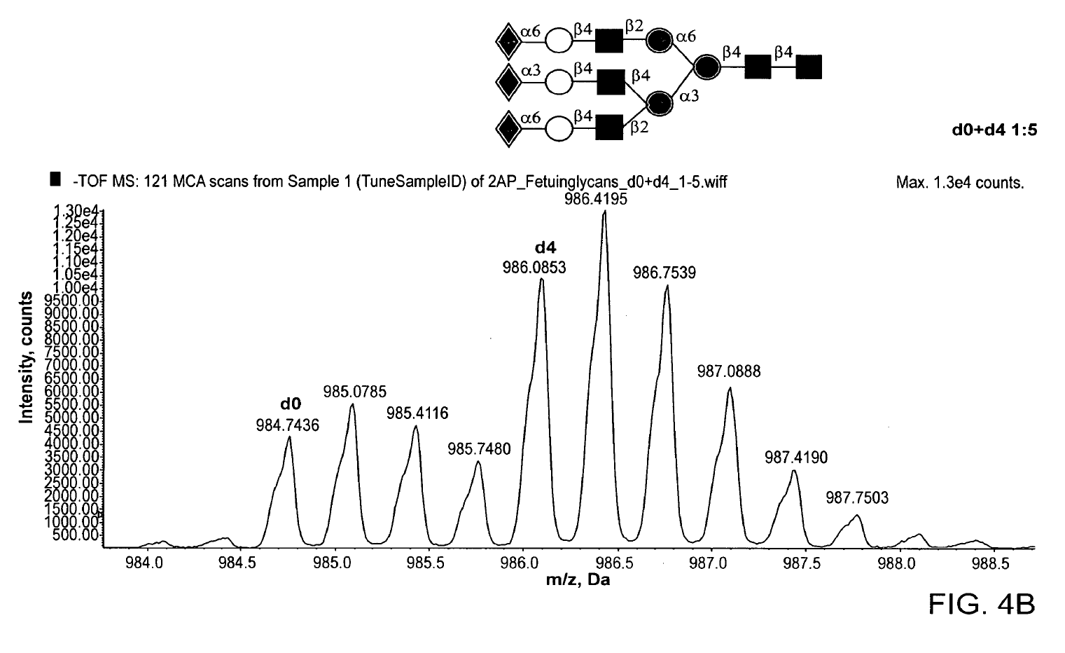

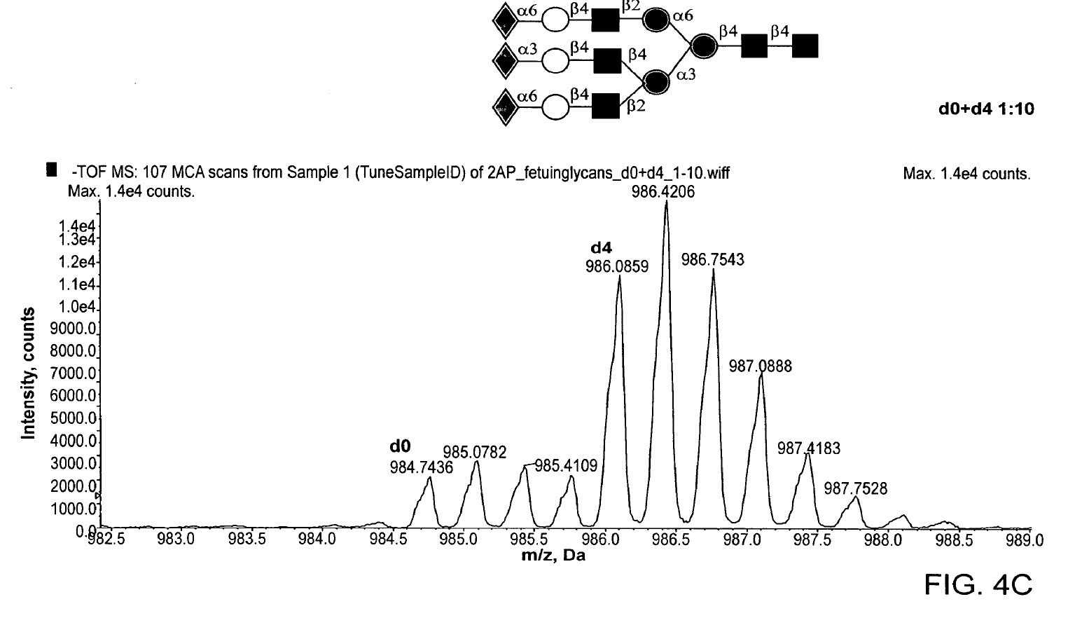

FIG. 4 shows an example for the relative quantitation of glycan species released form different glycoprotein samples using the method described in FIG. 2. The spectra show the relative amounts obtained for a specific sialylated N-linked glycan derived form two different protein samples when these are combined in approximately 1:1 ratio (top spectrum), 1:5 ratio (middle spectrum) and 1:10 ratio (bottom spectrum).

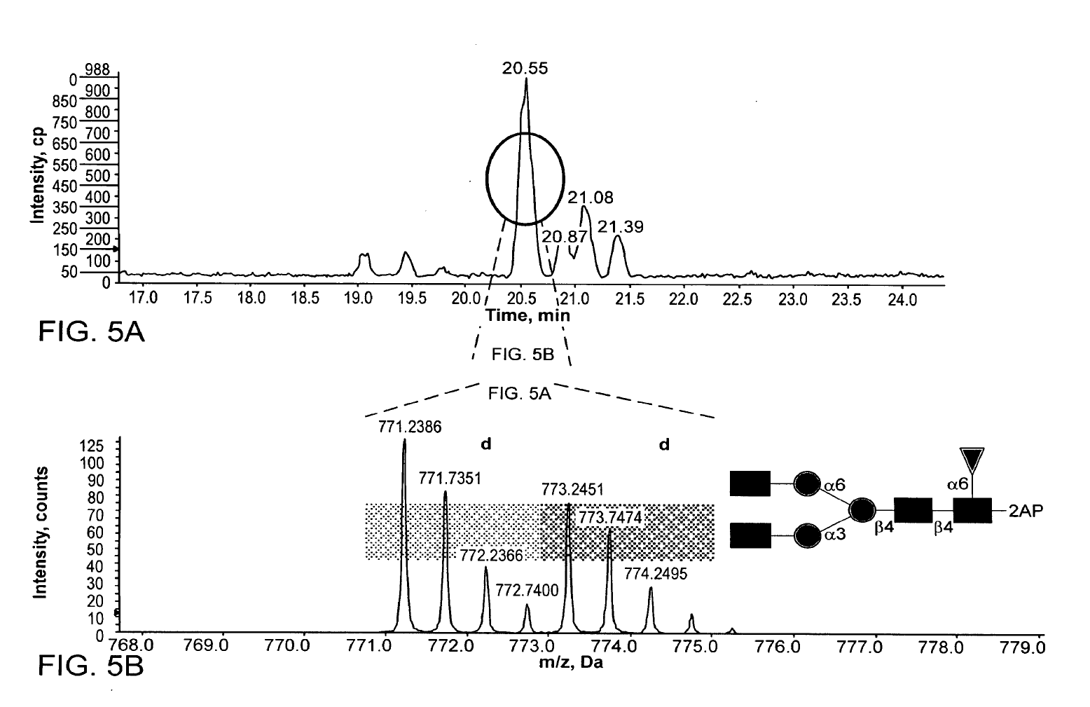

FIG. 5 shows examplary chromatographic and mass spectrometry data generated from glycan preparations according to scheme in FIG. 2. N-linked glycans were released from a model antibody glycoprotein (expressed under two different culture conditions) using N-Glycosidase F (PNGASE-F). The two resulting glycan samples were purified separately using activated graphitized carbon solid phase extraction cartridges. The glycan mixtures were individually labeled with 2-aminopyridine. One sample was modified to incorporate an isotopically-labeled 2-aminopyridine with 4 deuteriums (d₄) while the other sample was derivatized with a non-isotopically-labeled 2-aminopyridine (d₀). The samples were then mixed in a 1:1 ratio and analyzed by LC-MS using a graphitized carbon column. The chromatogram shown in FIG. 5A represents the separation of glycans species of the two combined samples containing d₀ and d₄-labeled glycans. The mass spectrum shown in FIG. 5B illustrates the relative quantitation of one particular glycan species present in the two samples based on the d₀/d₄ ratio.

### Equivalents

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the methods have been described in conjunction with various embodiments and examples, it is not intended that the present methods be limited to such embodiments or examples. On the contrary, the methods encompass various alternatives, modifications, and equivalents, as will be appreciated by those of skill in the art.

While the methods have been particularly shown and described with reference to specific illustrative embodiments, it should be understood that various changes in form and detail may be made without departing from the spirit and scope of the present disclosure. Therefore, all embodiments that come within the scope and spirit of the present disclosure, and equivalents thereto, are intended to be claimed. The claims, descriptions and diagrams of the methods, systems, and assays of the present disclosure should not be read as limited to the described order of elements unless stated to that effect.

1. A method of making an isotopically-labeled aminated glycan comprising reacting a glycan with an amine and an isotopically-labeled reducing agent to provide an isotopically-labeled aminated glycan.

2. The method according to claim 1, wherein the amine and the isotopically labeled reducing agent are reacted with the glycan simultaneously.

3. The method according to claim 1, wherein the amine and the isotopically labeled reducing agent are reacted with the glycan sequentially.

4. The method according to claim 1, wherein the isotopically-labeled reducing agent is deuterium (²H) labeled or tritium (³H) labeled.

5. The method according to claim 4, wherein the isotopically-labeled reducing agent is selected from the group consisting of NaBD₄, NABT₄, pyridine-BD₃, α-picoline-BD₂/AcOD; D₂-Pd/C, NaBD₃CN, NaBD(OCOCH₃)₃, and dialkylamino-BD₃.

6. (canceled)

7. The method according to claim 1, wherein the isotopically-labeled aminated glycan comprises at least one ²H atom.

8. The method according to claim 1, wherein the amine is an isotopically-labeled amine

9. The method according to claim 8, wherein the isotopically-labeled amine comprises at least one isotopic atom selected from the group consisting of ²H, ³H, ¹³C, ¹⁸O, ¹⁵N, ³³S, ³⁴S, ³²P, ²⁹Si, ³⁰S, ¹⁰B and ¹¹B.

10-14. (canceled)

15. The method according to claim 1, wherein the amine is a primary amine or ammonia (NH₃).

16. The method according to claim 15, wherein the primary amine is substituted or unsubstituted, cyclic or acyclic, branched or unbranched alkyl amine; substituted or unsubstituted, cyclic or acyclic, branched or unbranched alkenyl amine; substituted or unsubstituted, cyclic or acyclic, branched or unbranched alkynyl amine; substituted or unsubstituted, cyclic or acyclic, branched or unbranched heteroalkyl amine; substituted or unsubstituted aryl amine; or substituted or unsubstituted heteroaryl amine.

17-21. (canceled)

22. A method for determining the relative amount of a glycan species in at least two different glycan preparations, comprising steps of:
(i) reacting a first glycan preparation with a first amine and a first reducing agent, one of which is isotopically-labeled, to provide a first aminated, isotopically-labeled glycan preparation;
(ii) reacting a second glycan preparation with a second amine and a second reducing agent to provide a second aminated glycan preparation;
(iii) combining an amount of the first aminated, isotopically-labeled glycan preparation with an amount of the second aminated glycan preparation to provide a glycan mixture;
(iv) analyzing the glycan mixture by mass spectrometry to provide a mass spectrum which includes at least one pair of peaks that corresponds to aminated versions of a glycan species from the first and second aminated glycan preparations;
(v) determining the relative intensities of the peaks in the at least one pair of peaks; and
(vi) quantifying the relative amounts of the glycan species provided in the first and second glycan preparations in light of the relative intensities of the peaks.

23. The method according to claim 22, wherein the first reducing agent is an isotopically-labeled reducing agent.

24-27. (canceled)

28. The method according to claim 22, wherein the first amine is an isotopically-labeled amine that comprises at least one isotopic atom selected from the group consisting of ²H, ³H, ¹³C, ¹⁸O, ¹⁵N, ³³S, ³⁴S, ³²P, ²⁹Si, ³⁰S, ¹⁰B and ¹¹B.

29-34. (canceled)

35. The method according to claim 22, wherein the first amine is a primary amine or ammonia (NH₃).

36. The method according to claim 22, wherein the mass of the first amine differs from the mass of the second amine by a mass in the range of about 1 to 10 Daltons.

37. (canceled)

38. The method according to claim 22, wherein the first amine is an isotopically enriched analog of the second amine

39. The method according to claim 22, wherein the first and second amines are isotopically enriched.

40-48. (canceled)

49. The method according to claim 22, wherein the first glycan preparation is obtained from a first glycoprotein preparation, and the second glycan preparation is obtained from a second glycoprotein preparation, and the first and second glycoprotein preparations are obtained using the same cell culture conditions.

50. The method according to claim 22, wherein the first glycan preparation is obtained from a first glycoprotein preparation, and the second glycan preparation is obtained from a second glycoprotein preparation, and the first and second glycoprotein preparations are obtained using different cell culture conditions.

51-52. (canceled)

53. The method according to claim 22, wherein the method is used to assess glycosylation characteristics of cells or cell lines that are being considered for production of a particular glycoprotein.

54. The method according to claim 22, wherein the method is used to monitor a cell culture to assess the likelihood that it will generate a product with a glycosylation pattern as close to identical with the established glycosylation pattern of a pharmaceutical product.

55. A method of making an isotopically-labeled aminated glycan, comprising reacting a glycan with an isotopically-labeled amine and an isotopically labeled reducing agent to provide an isotopically aminated glycan.

56-57. (canceled)
